(19) United States
(12) Patent Application Publication
(10) Pub. No.: US 2004/0014240 A1
(43) Pub. Date: Jan. 22, 2004

(54) Molecule detecting sensor

(76) Inventors: Keigo Takeguchi, Fuji-shi (JP); Tsuneo Sato, Fuji-shi (JP); Teruaki Katsube, Tokyo (JP); Hidekazu Uchida, Tokyo (JP)

Correspondence Address: Finnegan Henderson Farabow, Garrett & Dunner, 1300 I Street NW, Washington, DC, 20005, US

(21) Appl. No.: 10/332,012

(22) PCT Filed: Jul. 6, 2001
(86) PCT No.: PCT/JP01/05917

(30) Foreign Application Priority Data
Jul. 6, 2000 (JP) 2000-205492
Apr. 27, 2001 (JP) 2001-133277

(51) Int. Cl. C12M 1/34; G01N 33/543

(52) U.S. Cl. 436/518; 435/287.2

(57) ABSTRACT

To improve photocurrent characteristics of a molecule detecting sensor, the molecule detecting sensor is formed in the steps of: depositing a first silicon layer (12) on a R-plane sapphire substrate (11); turning the first silicon layer (12) amorphous by implanting silicon ions thereinto in the vicinity of the silicon layer-sapphire substrate interface; recrystallizing an amorphous silicon layer by heat treatment; oxidizing part of the first silicon layer (12) by introducing a recrystallized silicon layer into an oxidation furnace; removing a silicon oxide film (13) formed by an oxidation; depositing a second silicon layer (15) on a seed silicon layer (14) which is a first silicon layer left after removing the steps of forming an insulator layer (3) on a single crystal silicon layer (2) which is a laminated structure made up of the seed silicon layer (14) and the second silicon layer (15); and placing an electrolyte (4) on the insulator layer.

### TECHNICAL FIELD

This invention relates to: a molecule detecting sensor which is designed to include a laminated structure made up of a semiconductor layer and an insulator layer superposed thereon and an electrolyte placed on the laminated structure and to detect molecules, such as genes, in the steps of illuminating the semiconductor layer with light and quantitatively determining the chemical state of the electrolyte by taking photocurrent induced in the semiconductor layer through the illumination of light; a method for manufacturing the same; and a method for detecting molecules.

### BACKGROUND ART

As a chemical sensor which is designed to include a laminated structure made up of a semiconductor layer and an insulator layer superposed thereon and an electrolyte placed on the laminated structure and to quantitatively determine the chemical state of the electrolyte in the steps of: illuminating the semiconductor layer with light from its back; and taking photocurrent induced in the semiconductor layer through the illumination of light, the one adopting the method of LAPS (Light Addressable Potentiometric Sensor) is well known.

The chemical sensor adopting the LAPS method consists of a semiconductor substrate/insulator layers and usually has a structure made up of: a single crystal silicon substrate, as a semiconductor substrate; a silicon oxide layer, as an insulator layer, obtained by subjecting a silicon substrate to thermal oxidation; and a silicon nitride layer formed on the silicon oxide layer to protect the same.

In the chemical sensor, an electrolyte as a sample is placed on the insulator layer, and the semiconductor substrate is illuminated with a beam of modulated light from its back. The characteristics of the photocurrent induced in the semiconductor substrate by the illumination of a beam of light vary depending on the charge in the electrolyte with which the semiconductor substrate comes in contact through the insulator layer, whereby the chemical state of the electrolyte such as pH can be quantitatively determined.

The use of such a chemical sensor permits the following things.

First, it permits quantitatively determining the local pH value of the electrolyte according to the position and area of the spot to which light is applied. In other words, considering the surface of the chemical sensor, that is, the surface of the electrolyte to be one face of a sample, the chemical variation in the spot in the sample's face to which light has been applied can be quantitatively determined. In this case, if light is concentrated with a condenser lens or the like, much more local information can be obtained.

Second, it permits obtaining image information from the pH values obtained by scanning the illumination light. In other words, considering the surface of the electrolyte to be one face of a sample, image information according to the chemical state of the electrolyte can be obtained by scanning light in the sample's face; and the chemical sensor can be used as a scanning chemical microscope. In this case, the spatial resolving power of the microscope depends on how much the spot area can be decreased when the illumination light is in the form of a beam.

Third, it permits detecting a wide variety of substances by forming a sensitive film, which causes a change in charge depending on the substance which is subject of measurement, on the insulator layer in contact with the electrolyte. For example, forming a silicon nitride film, as a sensitive film, on the insulator layer enables the detection of pH values. Forming a lipid film enables the sensor to be used as a taste sensor. And forming a platinum thin film enables the sensor to be used as a gas sensor.

Fourth, the sensor can be used as a multi-array sensor, though it is a single sensor, by arraying different sensitive films in one plane. For example, the sensor has been applied as a taste sensor in which different types of lipid films are fixed in one plane to specify types of taste from the measured patterns.

When illuminating the semiconductor substrate with a beam of light from its back as described above, however, if a bulk silicon substrate as thick as several hundreds (μm) is used, the carriers generated in the semiconductor substrate are lost due to their long-distance diffusion and do not reach the depletion layer even by the light illumination from the back of the semiconductor substrate, which makes it impossible to obtain photocurrent response.

Thus, a method has been adopted in which the semiconductor substrate is illuminated with light from the electrolyte side or the thickness of the bulk silicon substrate used is reduced to as thin as about 100 (μm).

When illuminating the semiconductor substrate from the electrolyte side, however, there a rise effects of, for example, light absorption by the electrolyte. And when illuminating the semiconductor substrate from its back, the use of a bulk silicon substrate as thin as about 100 (μm) has made it impossible to obtain photocurrent response unless light is used of which wavelength is as long as that of the infrared light, because the optical absorption coefficient of silicon increases as the wavelength of illumination light decreases.

Since the beam diameter of coherent light is proportional to its wavelength, the longer the wavelength, the larger the beam diameter, which decreases the spatial resolving power of the sensor when it is used as an image sensor. Thus, the upper limit of the spatial resolving power when using a silicon substrate 100 (μm) thick has been about 100 (μm).

Decreasing the thickness of the silicon substrate to less than 100 (μm) gives rise to a problem of being unable to maintain its mechanical strength, resulting in markedly limiting the mass of the samples to be measured.

In order to avoid this, there have been proposed a method in which an SOI substrate including a transparent insulator and a silicon layer formed thereon is used, as disclosed in, for example, Japanese Patent Publication No. 7-109414 Specification. Specifically, the method aims at obtaining sufficient mechanical strength of a sensor by forming an insulator layer and an electrolyte on the SOI substrate and improving the spatial resolving power of the same using a SOI substrate with a silicon layer having a thickness as small as 1 (μm) or less, since the method allows the thickness of the silicon layer to be set arbitrarily, and a light source of short wavelength.

However, even in cases where a chemical sensor adopting the LAPS method is constructed using the SOI substrate as above, the sensor only permits obtaining a faint photocurrent response and there remains a problem of being unable to obtain sufficient photocurrent response.

Particularly to improve the spatial resolving power of a scanning chemical microscope which uses such a chemical sensor or to increase the density of the array in a multi-array sensor which uses such a chemical sensor, the beam of illumination light need to be concentrated. But on the other hand, concentrating the illumination light decreases the amount of light entering the semiconductor substrate, which in turn decreases the amount of photocurrent induced, causing decrease in their capability as a sensor. To overcome this problem, improvement in photocurrent characteristics has been desired.

In recent years, technologies relating to gene-sequencing have been remarkably developed and the gene sequences of not only the human genome, but also the genomes of many organisms are being determined. To detect the expression, mutation, polymorphism, etc. of genes in a short period of time on the basis of such information, DNA microarrays, represented by DNA chips, which have many DNA molecules arrayed on their solid-phase carriers have been developed.

Such detection is conducted by using nucleic acids labeled with fluorescence, radioisotope (RI), or a compound for causing chemical reaction or, when labeling is not adopted, using compounds, referred to as intercalator, which combine with nucleic acids by the known techniques such as optical detection, electronic detection, radiation detection and chemical detection.

The labeling of nucleic acids as described above, however, requires complicated manipulations and, particularly when using fluorescence or RI, quick manipulations are required, taking into account the color degradation of fluorescent dyes or safety.

Thus, a detecting method has been desired which enables specified molecules such as nucleic acids to be detected more easily and more quickly.

This invention has been achieved in light of the above problems which the prior art has unsolved. Accordingly, an object of this invention is to provide: a molecule detecting sensor which has improved photocurrent characteristics and enables specified molecules to be detected more easily and more quickly; a method for manufacturing the same; and a method for detecting molecules.

### DISCLOSURE OF THE INVENTION

In order to accomplish the above object, this invention provides a molecule detecting sensor which is designed to include a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light, characterized in that the semiconductor layer is a single crystal silicon layer with a film thickness of 0.3 (μm) or more and 3.0 (μm) or less and that the full width at half maximum (FWHM) of the X-ray diffraction rocking curve of the (004) plane of the single crystal silicon is 1000 (arcsec) or less or the crystal defect density of the same is 1×10⁸ (cm⁻²) or less.

The film thickness of the single crystal silicon layer need not be exactly 0.3 (μm) or more and 3.0 (μm) or less and it can be on the order of 0.3 (μm) or more and on the order of 3.0 (μm) or less.

The FWHM of the X-ray diffraction rocking curve need not be exactly 1000 (arcsec) or less and it can be on the order of 1000 (arcsec) or less. And the crystal defect density can be on the order of 1×10⁸ (cm⁻²) or less.

Further, this invention provides a molecule detecting sensor which is designed to include a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light, characterized in that there exists heavily doped impurity in the semiconductor layer in the vicinity of the semiconductor layer-light transmitting substrate interface.

As the heavily doped impurity, desirably 1×10¹⁷ to 1×10²⁰ (cm⁻³) of impurity is added. However, the dopant dose need not be exactly 1×10^{17 to} 1×10²⁰ (cm⁻³) and it can be on the order of 1×10¹⁷ or more and on the order of 1×10²⁰ (cm⁻³) or less.

Desirably an antireflection film is provided between the semiconductor layer and the light transmitting substrate. Providing such an antireflection film can inhibit light from being reflected from the light transmitting substrate surface exposed to light, leading to improvement in photocurrent characteristics.

The thickness of the antireflection film can be set according to the wavelength of the illumination light. Specifically, since the reflectance of the antireflection film varies depending on the wavelength of the illumination light as well as the film thickness of the antireflection film, the reflection of the illumination light can be inhibited by setting the thickness of the antireflection film according to the wavelength of the illumination light.

The thickness of the semiconductor layer can be set according to the wavelength of the illumination light. Specifically, since the reflectance of the single crystal silicon layer varies depending on the wavelength of the illumination light as well as the film thickness of the single crystal silicon layer, the reflection of the illumination light can be inhibited by setting the film thickness of the single crystal silicon layer according to the wavelength of the illumination light.

Further, the relation between the depletion-layer spreading position and the light penetrating depth in the semiconductor layer which enables a large amount of photocurrent to be obtained can be realized by setting the thickness of the semiconductor layer according to the wavelength of the illumination light, since the amount of photocurrent induced depends on the relation between the depletion-layer spreading position and the light penetrating depth in the semiconductor layer.

As the light transmitting substrate, a single crystal oxide substrate or a glass substrate containing SiO₂ is applicable.

Desirably the thickness of the semiconductor layer is 10 (μm) or less. However, the thickness need not be exactly 10 (μm) or less and it can be on the order of 10 (μm) or less.

As the single crystal oxide substrate, a sapphire substrate is applicable.

Further, this invention provides a method for manufacturing a molecule detecting sensor which is designed to include a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light, characterized in that it includes: a film forming step of forming a first silicon layer on the light transmitting substrate; an oxidizing step of oxidizing exclusively the top portion of the first silicon layer by heat treatment in the oxidizing atmosphere; and a removing step of removing the silicon oxide film formed in the oxidizing step and an epitaxy step of growing a second silicon layer by epitaxy on the first silicon layer after the removing step and uses the laminated structure made up of the first silicon layer and second silicon layer as the semiconductor layer.

The method may further include a recrystallizing step, right after the film forming step, of recrystallizing the first silicon layer in the sub-steps of: implanting silicon ions into the first silicon layer to turn the same amorphous in the vicinity of the first silicon layer-light transmitting substrate interface; and heat treating the amorphous silicon layer.

Or the method may repeat the oxidizing, removing and epitaxy steps two times or more for the second silicon layer formed through the epitaxy step, considering the same as the first silicon layer in the oxidizing step, and use the laminated structure made up of the silicon layers thereby formed as the semiconductor layer.

Further, this invention provides a method for manufacturing a molecule detecting sensor which is designed to include a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light, characterized in that it includes: a film forming step of forming a first silicon layer on the light transmitting substrate; a first recrystallizing step of recrystallizing the first silicon layer in the sub-steps of implanting silicon ions into the first silicon layer to turn the same amorphous in the vicinity of the first silicon layer-light transmitting substrate interface and heat treating the amorphous silicon layer; an oxidizing step of oxidizing exclusively the top portion of the first silicon layer by heat treating the first silicon layer in the oxidizing atmosphere after the first recrystallizing step; a removing step of removing the silicon oxide film formed in the oxidizing step; an epitaxy step of growing a second silicon layer by epitaxy on the first silicon layer left after the removing step; and a second recrystallizing step of recrystallizing the laminated structure made up of the first silicon layer and second silicon layer in the sub-steps of implanting silicon ions into the laminated structure to turn the same amorphous in the vicinity of the laminated structure-light transmitting substrate interface and heat treating the amorphous laminated structure and that it uses the laminated structure of the silicon layers after the second recrystallizing step as the semiconductor layer.

The method may further include an epitaxy step of growing a third silicon layer by epitaxy on the second silicon layer after the second recrystallizing step and use the laminated structure made up of the silicon layers including the third silicon layer as the semiconductor layer.

The method may further include, after the second recrystallizing step, an oxidizing step of oxidizing exclusively the top portion of the second silicon layer by heat treating the same in the oxidizing atmosphere; a removing step of removing the silicon oxide film formed in the oxidation; and an epitaxy step of growing a third silicon layer by epitaxy on the second silicon layer left after the removing step and use the laminated structure made up of the silicon layers including the third silicon layer as the semiconductor layer.

Further, this invention provides a method for manufacturing a molecule detecting sensor which is designed to include a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light, characterized in that it includes the steps of: implanting hydrogen or rare gas ions into the surface of a single crystal silicon substrate to form an ion diffusion region of hydrogen or rare gas therein; bonding the ion diffusion region of the single crystal silicon substrate and the light transmitting substrate together; causing the single crystal silicon substrate to cleave in the ion diffusion region by heat treatment after the bonding step, to form a single crystal silicon layer on the light transmitting substrate; and planarizing the cleaved surface of the single crystal silicon layer by polishing and in addition, at least any one of the steps of: arranging a transparent conductive film between the semiconductor layer and the light transmitting substrate; arranging a heavily doped impurity in the semiconductor layer in the vicinity of the semiconductor layer-light transmitting substrate interface; forming an antireflection film between the semiconductor layer and the light transmitting substrate; and forming an antireflection film on the light transmitting substrate surface exposed to light and it uses the planarized single crystal silicon layer as the semiconductor layer.

Still further, this invention provides a method for manufacturing a molecule detecting sensor which is designed to include a semiconductor layer and an insulator layer stacked in this order on a light transmitting substrate an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light, characterized in that it includes the steps of: anodizing the surface of a single crystal silicon substrate to form a porous single crystal silicon layer; annealing the porous single crystal silicon layer in the hydrogen atmosphere to make the uppermost surface of the same a single crystal silicon layer on which another single crystal silicon layer is grown by epitaxy; bonding the light transmitting substrate to the surface of the grown single crystal layer; removing the single crystal silicon substrate and the porous single crystal silicon layer; and planarizing the surface of the single crystal silicon layer having been exposed after the removing step and that it uses the planarized single crystal silicon layer as the semiconductor layer.

The method may include a step of arranging a transparent conductive film between the semiconductor layer and the light transmitting substrate.

The method may also include a step of arranging a heavily doped impurity in the vicinity of the semiconductor layer-light transmitting substrate interface.

The method may also include a step of forming an antireflection film between the semiconductor layer and the light transmitting substrate.

The method may also include a step of forming an antireflection film on the light transmitting substrate surface to be exposed to light.

The thickness of the antireflection film can be set according to the wavelength of the illumination light.

As the light transmitting substrate, a single crystal oxide substrate or a glass substrate containing SiO₂ is applicable.

As the single crystal oxide substrate, a sapphire substrate is applicable.

Further, this invention provides a molecule detecting sensor which is designed to include a semiconductor layer and an insulator layer stacked in this order and a plurality of sensitive films formed on the insulator layer and to detect molecules in the steps of: illuminating the semiconductor layer with light from its back while keeping an electrolyte placed on the sensitive films; detecting the photocurrent induced in the semiconductor layer through the illumination of light using electrodes provided in the electrolyte as well as on the semiconductor layer; and quantitatively determining the electrolyte based on the detected photocurrent, characterized in that the plurality of sensitive films are positioned so that they are the same distance from the electrode provided on the semiconductor layer.

Desirably, the electrode on the semiconductor layer is provided around the periphery of the region corresponding to the lower part of the electrolyte of the semiconductor layer and the sensitive films are provided at the positions which are the same distance from the electrode provided on the semiconductor layer in a plurality of linear arrays.

In a semiconductor layer such as a single crystal silicon layer in the form of a thin film, its resistance becomes higher at positions more distant from the electrode provided thereon. Therefore, the photocurrent detected for the electrolyte, which is expected to be constant, does not become constant even if the characteristics of the electrolyte, such as pH value, are uniform, due to the positional dependency of the resistance in semiconductor. And the characteristics of the electrolyte can sometimes be misjudged to be nonuniform; in actuality, it is uniform though.

However, if the sensitive films are positioned so that they are the same distance from the electrode, since the resistance values of the semiconductor at these positions are the same, changes in photocurrent characteristics due to the positional dependency of the resistance can be avoided.

This can be realized easily by: for example, removing the insulator layer leaving the portion for the circular electrolyte region, on which an electrolyte is to be placed, at the center of the laminated structure made up of the light transmitting substrate, semiconductor layer and insulator layer; forming a taking electrode around the periphery of the electrolyte region on the semiconductor layer; and arranging sensitive films on the periphery of an figure, which is analogous and concentric with the opening of the taking electrode, on the electrolyte placed in the opening of the taking electrode.

Further, this invention provides a molecule detecting sensor including: an insulator layer superposed on a semiconductor layer; a molecule probe which is fixed on the insulator layer and combines with a specific molecule; an electrolyte arranged on the insulator layer containing at least the molecule probe; and electrical characteristic detecting means for detecting electrical characteristics induced by stimulating the semiconductor, characterized in that it detects the specific molecule in the electrolyte by detecting the changes in the electrical characteristics due to the bonding of the molecule probe to the specific molecule in the electrolyte.

In the molecule detecting sensor, the semiconductor layer may be provided on a carrier substrate.

As the specific molecule, nucleic acids or the derivatives thereof are applicable.

Further, in the molecule detecting sensor, a plurality of molecule probes as above may be arranged on the insulator layer. The plurality of molecule probes may combine with different specific molecules or with the same specific molecule.

The molecule detection may be performed based on the results of the differential measurements made for the sites where the molecule probe exists and where no molecule probe exists.

In the molecule detecting sensor, the electrical characteristic detecting means may be constructed so that it applies an electric field between the electrolyte and the semiconductor layer to bond the molecule probe and the specific molecule together and applies an electric field again to release the molecule probe and the specific molecule from their incomplete bonding due to the first application of electric field.

In the molecule detecting sensor, a bonding molecule which can combine with the specific molecule, but is different from the molecule probe may be introduced into the electrolyte.

As the bonding molecule, an intercalator is applicable.

Or, as the bonding molecule, a protein which can combine with a nucleic acid or the derivative thereof is also applicable.

Desirably, the protein is an antibody which can combine with a nucleic acid or the derivative thereof.

As the bonding molecule, a nucleic acid or the derivative thereof is also applicable.

The bonding molecule may be modified with urease.

The bonding molecule may be modified with ferrocene.

As the carrier substrate, a light transmitting substrate is applicable.

As the semiconductor layer, a single crystal silicon layer is applicable.

In the single crystal silicon layer, desirably the FWHM of the X-ray diffraction rocking curve of its (004) plane is 1000 (arcsec) or less and its crystal defect density is 1×10⁸ (cm⁻²) or less.

However, the FWHM of the X-ray diffraction rocking curve need not be exactly 1000 (arcsec) or less and it can be on the order of 1000 (arcsec) or less. And the crystal defect density can be on the order of 1×10⁸ (cm⁻²) or less.

In the single crystal silicon layer, desirably the surface roughness is 4 (nm) or less. However, the surface roughness need not be exactly 4 (nm) or less and it can be on the order of 4 (nm) or less.

The thickness of the semiconductor layer can be set according to the wavelength of the illumination light.

And desirably the thickness of the semiconductor layer is 10 (μm) or less. However, the thickness of the semiconductor layer need not be exactly 10 (μm) or less and it can be on the order of 10 (μm) or less.

In the molecule detecting sensor, there may exist heavily doped impurity with a concentration of 1×10¹⁷ to 1×10²⁰ (cm⁻³) in the semiconductor layer in the vicinity of the semiconductor layer-light transmitting substrate interface.

Or a transparent conductive film may be provided between the semiconductor layer and the light transmitting substrate.

Further, an antireflection film may be provided at least either between the semiconductor layer and the light transmitting substrate or on the light transmitting substrate surface exposed to light.

The thickness of the antireflection film can be set according to the wavelength of the illumination light.

As the carrier substrate and the semiconductor layer, an SOS substrate consisting of a sapphire single crystal substrate and a single crystal silicon layer may be applied.

Further, as the carrier substrate, an electro-conductive substrate may be applied.

As the stimulation, a modulated electromagnetic wave may be applied.

Or it is also possible to use a modulated electric signal as the stimulation and an insulating substrate as the carrier substrate.

Further, this invention provides a method for detecting molecules, characterized in that it detects a specific molecule in an electrolyte with a molecule detecting sensor, including a semiconductor layer and an insulator layer stacked in this order on a carrier substrate, a molecule probe, which combines with the specific molecule, fixed on the insulator layer and an electrolyte arranged on the insulator layer which includes at least the molecule probe, by: detecting electric characteristics induced through the stimulation of the semiconductor layer; and detecting the changes in the electric characteristics due to the bonding of the molecule probe to the specific molecule in the electrolyte.

Further, this invention provides a method for detecting molecules, characterized in that it detects a specific molecule in an electrolyte with a molecule detecting sensor, including a semiconductor layer and an insulator layer stacked in this order on a carrier substrate, a molecule probe, which can combine with the specific molecule, fixed on the insulator layer and an electrolyte arranged on the insulator layer which includes at least the molecule probe, by: introducing a bonding molecule which can combine with the specific molecule but is different from the molecule probe; detecting electric characteristics induced through the stimulation of the semiconductor layer; and detecting the changes in the electric characteristics due to the bonding of the molecule probe to the specific molecule in the electrolyte as well as the bonding of the specific molecule to the bonding molecule.

Further, this invention provides a molecule detecting sensor which is designed to include: an insulator layer superposed on a semiconductor layer; a molecule probe which is fixed on the insulator layer and combines with a specific molecule; an electrolyte placed on the insulator layer which includes at least the molecule probe; and electric characteristic detecting means for detecting the electric characteristics induced through the stimulation of the semiconductor layer and to detect the specific molecule in the electrolyte by detecting the changes in the electric characteristics due to the bonding of the molecule probe to the specific molecule in the electrolyte, characterized in that the stimulation of the semiconductor layer is provided from the electrolyte side.

In the molecule detecting sensor, as the specific molecule, a nucleic acid or the derivative thereof is applicable.

Further, in the molecule detecting sensor, a plurality of molecule probes as above may be arranged on the insulator layer. The plurality of molecule probes may combine with different specific molecules or to the same specific molecule.

The molecule detection may be performed based on the results of the differential measurements made for the sites where the molecule probe exists and where no molecule probe exists.

In the molecule detecting sensor, the electrical characteristic detecting means may be constructed so that it applies an electric field between the electrolyte and the semiconductor layer to bond the molecule probe and the specific molecule together and applies an electric field again to release the molecule probe and the specific molecule from their incomplete bonding due to the first application of electric field.

In the molecule detecting sensor, a bonding molecule which can combine with the specific molecule, but is different from the molecule probe may be introduced into the electrolyte.

As the bonding molecule, an intercalator is applicable.

Or, as the bonding molecule, a protein which can combine with a nucleic acid or the derivative thereof is also applicable.

As the protein, an antibody which can combine with a nucleic acid or the derivative thereof is applicable.

As the bonding molecule, a nucleic acid or the derivative thereof is also applicable.

The bonding molecule may be modified with urease.

The bonding molecule may be modified with ferrocene.

### BRIEF DESCRIPTION OF THE DRAWINGS

FIG. 1 is a schematic configuration showing one example of molecule detecting sensors to which this invention is applied;

FIG. 2 is a schematic configuration showing another example of molecule detecting sensors according to this invention;

FIGS. 3(a) to 3(d) are cross-sectional views showing part of a production process of an SOI substrate in accordance with a first embodiment of this invention;

FIG. 4 is a graph showing results of measuring an X-ray diffraction rocking curve of a single crystal silicon layer in accordance with the first embodiment of this invention;

FIG. 5 is a graph showing results of measuring Surface Photo-Voltage (SPV) characteristics of the single crystal silicon layer in accordance with the first embodiment of this invention;

FIG. 6 is a characteristic diagram showing a correspondence between a film thickness of a single crystal silicon layer and a maximum photocurrent;

FIG. 7 is a graph showing results of measuring SPV characteristics for the single crystal silicon layer different from that of FIG. 6 in film quality;

FIG. 8 is a configuration showing an SOI substrate in accordance with the first embodiment when it is provided with an antireflection film;

FIGS. 9(a) to 9(c) are characteristic diagrams showing a correspondence between a film thickness of antireflection films and a reflectance;

FIG. 10(a) is a diagrammatic view illustrating a positional dependency of resistance values in a single crystal silicon layer and FIG. 10(b) is a cross-sectional view of one example of SOI substrates of which a single crystal silicon layer 2 is provided with a heavily doped impurity layer 2a;

FIGS. 11(a) to 11(h) are cross-sectional views showing part of a production process of an SOI substrate in accordance with a second embodiment of this invention;

FIGS. 12(a) to 12 (d) are cross-sectional views showing part of a production process of an SOI substrate in accordance with a third embodiment of this invention;

FIG. 13 is a schematic configuration showing one example of molecular detecting sensors to which the SOI substrate in accordance with the third embodiment is applied;

FIGS. 14(a) to 14(e) are cross-sectional views showing part of a production process of an SOI substrate in accordance with a fourth embodiment of this invention;

FIG. 15 is a schematic configuration showing one example of scanning sensors formed using a molecule detecting sensor of this invention;

FIG. 16 is a schematic configuration showing one example of array sensors constructed by using molecule detecting sensors of this invention;

FIGS. 17(a) to 17(c) are diagrammatic views illustrating positions of individual sensors arranged at the time of constructing an array sensor by using molecule detecting sensors of this invention;

FIG. 18 is a schematic configuration showing another example of molecule detecting sensors to which this invention is applied;

FIG. 19 is a diagrammatic view illustrating an operation of another example of molecule detecting sensors to which this invention is applied;

FIG. 20 is a schematic configuration showing another example of molecule detecting sensors to which this invention is applied;

FIG. 21 is a schematic configuration showing another example of molecule detecting sensors to which this invention is applied;

FIG. 22 is a schematic configuration showing another example of molecule detecting sensors to which this invention is applied;

FIG. 23 is a cross-sectional view showing a molecule detecting sensor part which is used for a multi-array sensor;

FIG. 24 is a top view showing the molecule detecting sensor part which is used for a multi-array sensor;

FIG. 25 is a schematic configuration showing one example of the molecule detecting sensors used for measuring photocurrent values;

FIG. 26 is a top view of the molecule detecting sensor of FIG. 25;

FIG. 27 is a schematic block diagram showing one example of photocurrent signal processing portions used for the measurement of photocurrent;

FIG. 28 is a voltamograph at a position where no DNA probe exists;

FIG. 29 is a voltamograph at a position where DNA probes exist;

FIG. 30 is one example of the voltamographs obtained when a DNA is hybridized using an SOS substrate of which silicon layer is 2 (μm) thick;

FIG. 31 is one example of the voltamographs obtained when a DNA is hybridized at a position where no DNA probe exists using a bulk silicon substrate;

FIG. 32 is one example of the voltamographs obtained when a DNA is hybridized at a position where a DNA probe exists using a bulk silicon substrate;

FIG. 33(a) is a cross-sectional view showing a sensor part of a molecule detecting sensor using a bulk silicon substrate and FIG. 33(b) is a bottom view of the same;

FIG. 34 is a top view showing the sensor part of a molecule detecting sensor using a bulk silicon substrate;

FIG. 35 is a schematic configuration showing one example of the molecule detecting sensors using a bulk silicon substrate which is used for measuring photocurrent values;

FIG. 36 is a diagrammatic view illustrating a state in which photocurrent is induced in a molecule detecting sensor using a bulk silicon substrate;

FIG. 37 is a diagrammatic view illustrating a state in which photocurrent is induced in a molecule detecting sensor using an SOS substrate;

FIG. 38 is a schematic configuration showing one example of the molecule detecting sensors using a bulk silicon substrate; and

FIG. 39 is a schematic configuration showing another example of the molecule detecting sensors using a bulk silicon substrate.

### BEST MODE FOR CARRYING OUT THE INVENTION

Next, preferred embodiments of this invention will be described.

First, a first embodiment will be described giving several examples.

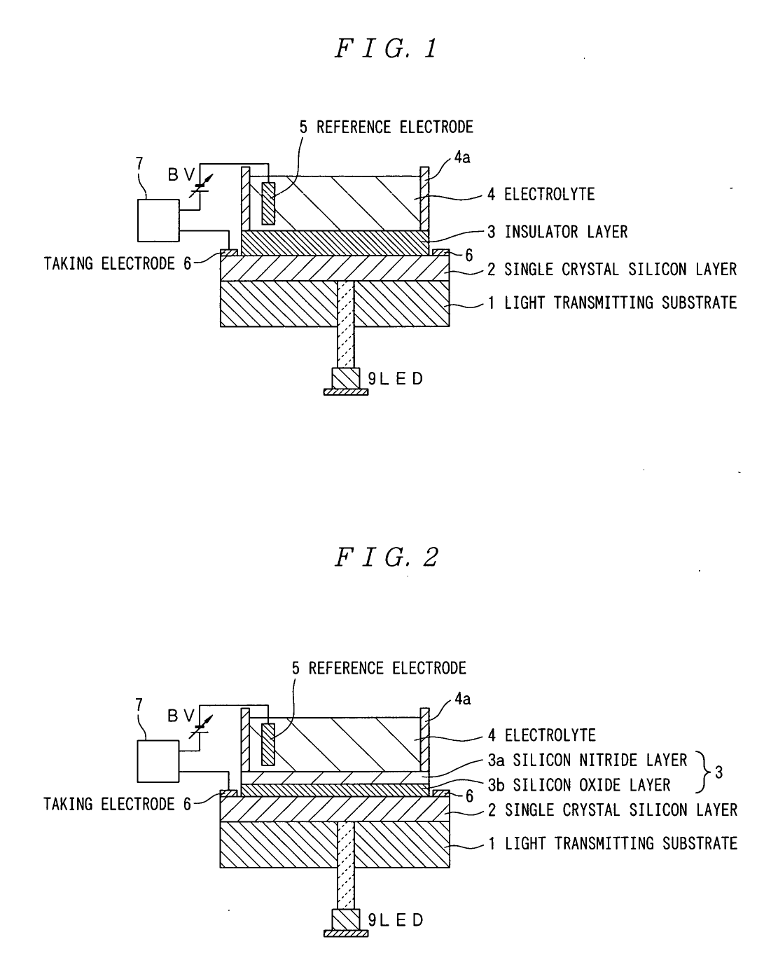

FIG. 1 is a schematic configuration showing one example of molecule detecting sensors to which this invention is applied.

In the same drawing, reference numeral 1 denotes a light transmitting substrate, reference numeral 2 a single crystal silicon layer superposed on the light transmitting substrate 1, reference numeral 3 an insulator layer superposed on the single crystal silicon layer 2, reference numeral 4 an electrolyte superposed on the insulator layer 3. Reference numeral 4a denotes a container formed on the insulator layer 3 to contain the electrolyte 4.

Reference numeral 5 denotes a reference electrode, numeral 6 a taking-out electrode, reference character BV a bias voltage, and reference numeral 7 a photocurrent signal processing portion. The chemical changes of the electrode 4 are detected by providing the single crystal layer with a light illumination pulse through the back of the light transmitting substrate 1 using a light emitting diode (LED) 9 and taking out changes in alternating photocurrent by the photocurrent signal processing portion 7 due to the light-induced carriers in the interface of the semiconductor layer 2 and the insulator layer 3.

As the light transmitting substrate 1, a single crystal oxide substrate, in particular a sapphire substrate is preferably used. This is because the sapphire substrate enables a single crystal silicon film of high quality to be grown by epitaxy relatively easily.

As the light transmitting substrate, glass substrates consisting of amorphous materials such as, for example, SiO₂ and plastic substrates consisting of, for example, a polycarbonate resin are also applicable. In this case, the interface of the light transmitting substrate and the single crystal silicon film to be bonded together may be a light transmitting insulator layer of, for example, silicon oxide or vacancy.

The surface of the light transmitting substrate to be illuminated with light is preferably polished to be flat.

The single crystal silicon layer 2 is desirably of high quality, which is specified by crystallinity, defect density and surface flatness. Specifically, in the single crystal silicon layer 2, desirably the FWHM of the X-ray diffraction rocking curve of its (004) plane is 1000 (arcsec) or less or its crystal defect density is 1×10⁸ (cm⁻²) or less. Desirably the surface roughness is 4 (nm) or less. And desirably the film thickness is 1 (nm) or more and 1×10⁵ (nm) or less, preferably 10 (nm) or more and 1×10⁴ (nm) or less.

As to the polarity of the silicon, it may be either n type or p type.

Silicon has a problem of being of high resistance in the thin film state, and the father it becomes away from electrodes, the more it makes it difficult for current to pass therethrough; accordingly, to avoid the problem, preferably a high concentration of impurity is implanted into the single crystal silicon layer in the vicinity of the single crystal silicon layer—light transmitting substrate interface. As the high concentration of impurity, either n type or p type can be used.

The dosage of the impurity in the ion implantation should be about 1×10¹⁷ to 1×10²⁰ (cm⁻³), preferably about 1×10¹⁹ (cm⁻³), because undue ion implantation causes the crystallinity to be ruined.

As one of the alternatives, for example, the single crystal silicon layer may be formed on a high-concentration-impurity layer which has been formed earlier.

As the insulator layer 3, is used a silicon oxide layer obtained by heating the single crystal silicon layer 2 in oxygen or steam, or silicon oxide chemically formed by dipping the single crystal silicon layer 2 into an acid or the like. However, for silicon oxide, its electrical properties are unstable once it is dipped into an electrolyte; therefore, preferably an insulator layer, such as silicon nitride layer 3a, aluminium oxide layer or tantalum oxide layer, is formed on the silicon oxide layer 3b, as shown in FIG. 2, or the dielectric as above maybe directly formed on the single crystal silicon layer 2. Desirably the total thickness of these insulator layers is 500 (nm) or less, preferably 100 (nm) or less.

To take the above-described photocurrent signal, the taking electrode 6 is formed which directly contact with the silicon layer at portions out of contact with the electrolyte 4. As the material for the taking electrode, any materials can be used as long as they are capable of having an ohmic contact with silicon, and usually aluminium or alloys containing aluminium as a major constituent are used. In this case, to strengthen the electrodes' adhesion to silicon, Ti or Cr may be inserted between the taking electrode 6 and the silicon layer.

Then the electrolyte 4 to be measured is placed in such a manner as to come in contact with the surface of the insulator layer 3 and electrodes are provided. As the electrodes, desirably three-electrode system is used, and the reference electrode 5 and the counter electrode (not shown in the drawing) are dipped into the electrolyte 4. As the reference electrode is used a saturated KCL-silver-silver chloride electrode, and as the counter electrode is used a platinum electrode in the form of a plate or a coil.

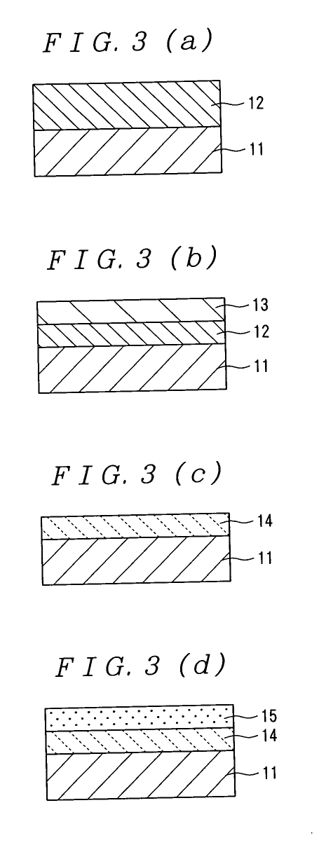

FIGS. 3(a) to 3(d) are cross-sectional views showing a production process of SOS substrate consisting of the light transmitting substrate 1 and the single crystal silicon layer 2, where a sapphire substrate is used as the light transmitting substrate.

Specifically, a 280 nm thick first silicon layer 12 was deposited on an R-plane sapphire substrate 11 by LPCVD using monosilane (SiH₄) as source gas material at a growth temperature of 950° C. (FIG. 3(a)).

Then, the first silicon layer was turned amorphous on the silicon layer 12—sapphire substrate 1 interface side by implanting 1×10¹⁶ (cm⁻²) silicon ions having an energy of 190 (KeV) thereinto while keeping the substrate temperature at 0 (° C.), and heat treated in nitrogen gas atmosphere at 550 (° C.) for 1 hour. Subsequently to this, the first silicon layer 12 was heat treated in the same atmosphere at a temperature raised to 900 (° C.) for 1 hour to be recrystallized.

The sapphire substrate 11 was then introduced into an oxidation furnace and steam oxidized under the condition of 1000 (° C.) for 30 minutes while introducing 180 (L/min) of hydrogen and 180 (L/min) of oxygen (FIG. 3(b)).

Successively, the sapphire substrate 11 was dipped into buffered hydrofluoric acid (BHF) to remove the silicon oxide layer 13 formed on the top of the first silicon layer 12 (FIG. 3(c)). The measured value of the thickness of a seed silicon layer 14, which was the first silicon layer after removing the silicon oxide layer 13, was 100 (nm).

Then, a second silicon layer 15 was grown by epitaxy on the seed silicon layer 14 by a UHV-CVD using monosilane (SiH₄) as source gas material at a growth temperature of 750° C. (FIG. 3(d)). After the epitaxial growth, the measured value of the total silicon film thickness of the seed silicon layer 14 and the second silicon layer 15 was 280 (nm).

For the SOS substrate formed in this manner, its orientation and crystallinity were evaluated with a HR-XRD (high resolution quateraxial X-ray diffractometer) using Kal ray of Cu as a source.

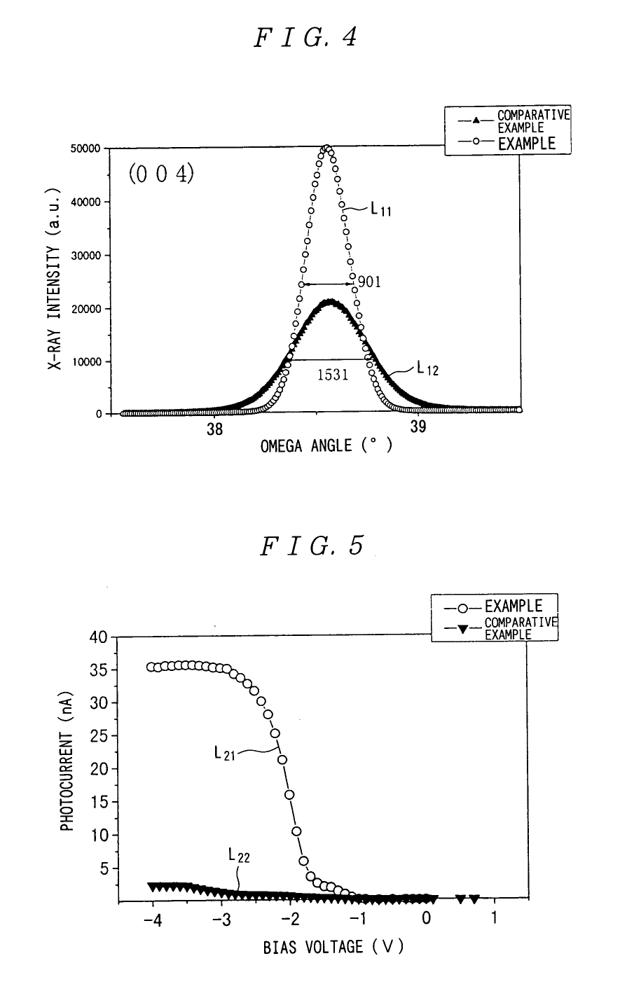

At the time of measuring the XRC (X-ray diffraction rocking curve) of the Si (004) plane grown parallel to the sapphire substrate 11, the FWHM was 901 (arcsec), as shown in the characteristic curve L₁₁ of FIG. 4.

In FIG. 4, X-ray intensity (a.u.) is plotted in ordinate and omega angle (°) in abscissa.

Successively, in order to measure the SPV of the SOS substrate, the SOS substrate was introduced into the oxidation furnace and steam oxidized under the condition of 1000 (° C.) for 10 minutes while introducing 180 (L/min) of hydrogen and 180 (L/min) of oxygen.

The film thickness of the oxide formed in this manner was 50 (nm).

Then, the oxide was etched in such a manner as to allow the oxide in the form of a circle 16 (mmφ) in diameter to remain at the center of the SOS substrate and Al film with thickness 100 (nm), as a taking electrode, was formed around the circular oxide by vapor deposition.

The container 4a was filled with the electrolyte solution, the reference electrode and the counter electrode were dipped into the solution, a shielding plate with a window 0.6 (mmφ) in diameter opened at its center is bonded to the surface of the SOS substrate to be illuminated with light, and blue LED 9 with wavelength 470 (nm) as illuminating light was flickered.

Then, the photocurrent was measured which flowed the external circuit connected to the reference electrode and the taking electrode. The characteristic curve L₂₁ of FIG. 5 shows the measured results. In the same figure the measured photo current (nA) is plotted in ordinate and the bias voltage (V) applied to the SOS substrate in abscissa.

X-ray diffraction rocking curve and photocurrent were measured in the same manner for the SOS substrate, as a comparative example, formed as follows.

In the SOS substrate of the comparative example, a 280 nm thick first silicon layer 12 was deposited on an R surface sapphire substrate by LPCVD using monosilane (SiH₄) as source gas material under the condition of 950 (° C.).

Then, the first silicon layer was turned amorphous on the silicon layer—sapphire substrate interface side by implanting 1×10¹⁶ (cm⁻²) silicon ions having an energy of 190 (KeV) thereinto while keeping the substrate temperature at 0 (° C.), and heat treated in nitrogen gas atmosphere at 550 (° C.) for 1 hour. Subsequently to this, the first silicon layer was heat treated in the same atmosphere at a temperature raised to 900 (° C.) for 1 hour to be recrystallized.

For the SOS substrate of the comparative example formed in this manner, its orientation and crystallinity were evaluated with a HR-XRD (high resolution quateraxial X-ray diffractometer) in the same manner as above; as a result, the FWHM of XRC of Si (004) plane was 1531 (arcsec), as shown in the characteristic curve L₁₂ of FIG. 4.

And when measuring the SPV characteristics of the SOS substrate in the same manner as above, the results were obtained as shown in the characteristic curve L₂₂ of FIG. 5.

As shown in FIG. 5, it was ascertained that the value of the saturation photocurrent of the SOS substrate according to this invention was 10 times or more as large as that of the SOS substrate of the comparative example.

Further, it is apparent that while the photocurrent value increased very slowly in the SOS substrate of the comparative example, the rising of photocurrent in the SOS substrate according to this invention was improved.

Accordingly, a molecule detecting sensor using the SOS (SOI) substrate obtained by such construction, which includes a silicon layer of high quality, enables the SPV characteristics to be improved due to the high-quality silicon layer, which means improvement in S/N ratio, leading to improvement in its sensitivity. Further, due to the increase in photocurrent amount, the illumination light can be concentrated, the spatial resolving power as an LAPS can be improved, and high sensitivity and high stability can be ensured.

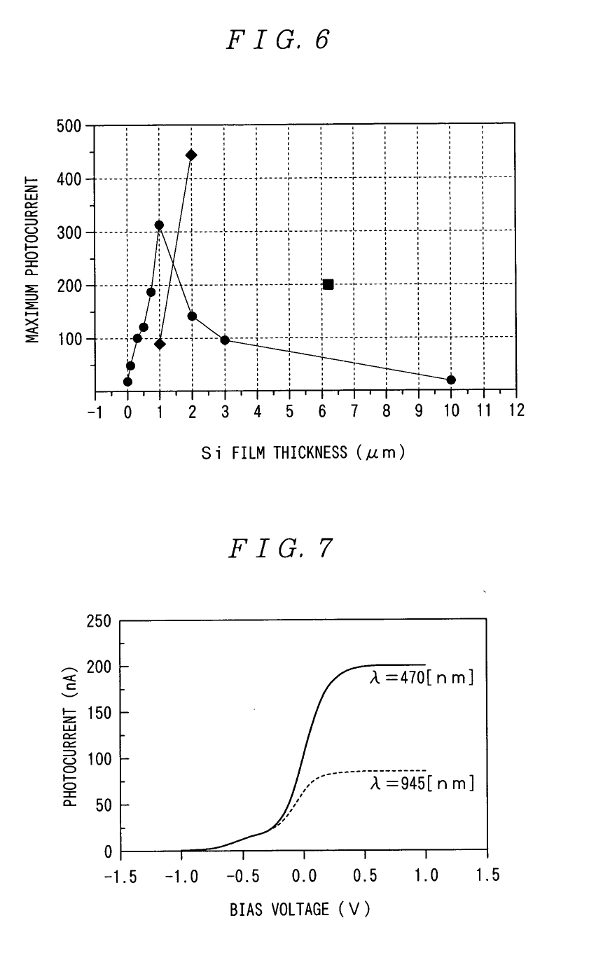

FIG. 6 is a characteristic diagram showing the results of measuring SPV characteristics at the time of varying the film thickness and quality of the silicon layer. In the same drawing, the film thickness (μm) of the single crystal silicon layer 2 is plotted in abscissa and the maximum photocurrent amount (nA) in ordinate.

In FIG. 6, ● represents the amount of the maximum photocurrent in the SPV characteristics at a wavelength of incident light of 470 (nm) when varying the film thickness of the single crystal silicon layer 2 in the SOS substrate, which are formed through the production process shown in FIG. 3, as follows: 50 (nm), 100 (nm), 300 (nm), 500 (nm), 750 (nm), 1000 (nm), 2000 (nm), 3000 (nm) and 10000 (nm).

The single crystal silicon layers 2 with different thickness as above were obtained by varying the thickness of the second silicon layer 15 with the length of time that the second silicon layer 15 is grown by epitaxy. Then a silicon oxide film with thickness 50 (nm) was formed on each of the single crystal silicon layers 2. As an electrolyte, 10 (mmol/l) of HEPES was used.

In FIG. 6, ♦ represents the amount of the maximum photocurrent in the SPV characteristics when stacking, as insulator layers, a silicon oxide film with thickness 50 (nm) and a silicon nitride with thickness 50 (nm) in this order on each of the single crystal silicon layers 2 with thickness 1000 (nm) and 2000 (nm) on the SOS substrate formed through the production process shown in FIG. 3. As an electrolyte, 10 (mmol/l) of TE buffer was used.

In FIG. 6, ▪ represents the amount of the maximum photocurrent in the SPV characteristics when superposing a silicon oxide film with thickness 50 (nm) on the SOS substrate which has a p-channel heavily doped layer with thickness 100 (nm) formed in the vicinity of the silicon-sapphire interface and of which silicon layer is about 6000 (nm) thick. As an electrolyte, 10 (mmol/l) of HEPES was used.

In the mean time, FIG. 7 is a characteristic diagram showing the results of measuring SPV characteristics for an SOS substrate different from the SOS substrate formed through the production process shown in FIG. 3 in film quality of the silicon layer.

This SOS substrate was formed through the following procedures.

First, a silicon layer 100 (nm) thick was grown on a seed silicon layer 110 (nm) thick by a LPCVD using monosilane as source gas material while introducing diborane so that borone concentration became 1019 (cm⁻³). Then, a silicon layer 6 (μm) thick was grown using monosilane gas alone as a raw material while cutting off diborane, to obtain an SOS substrate of which silicon layer was 6.21 (μm) thick and which had a P-type region heavily doped with boron in the vicinity of the silicon-sapphire interface.

In FIG. 7, bias voltage (V) is plotted in abscissa and photocurrent (nA) in ordinate. In the same figure, the solid and broken lines show the SPV characteristics when the wavelengths of incident light were 470 (nm) and 945 (nm), respectively.

As shown in FIG. 7, even when the silicon film thickness was about 6.21 (μm), photocurrent as much as about 200 (nA) could be obtained by forming a heavily doped layer in the vicinity of the silicon-sapphire interface.

As shown by ● in FIG. 6, it is apparent that in varying the film thickness, the maximum photocurrent reaches a peak when the thickness of the single crystal silicon layer 2 is 1000 (nm). This indicates that the more the thickness of the single crystal silicon layer 2 increases, the more the amount of optical absorption increases, and the reason the maximum photocurrent begins to decrease when the thickness becomes 1000 (nm) or more is possibly that the thickness exceeds the diffusion length of light.

Accordingly, it is known that when the wavelength of incident light is 470 (nm), preferably the thickness of the single crystal silicon layer 2 is 0.3 to 3 (μm).

However, as shown by ♦ in FIG. 6, it is apparent that photocurrent largely depends on the quality of the insulating film. And as shown by ▪ in FIG. 6 and in FIG. 7, it is also apparent that the responsiveness of photocurrent can be improved by the film quality or impurity concentration of the silicon layer 2.

As described above, even if the thickness of the silicon layer 2 is outside the range of 0.3 to 3 (μm), adequate photocurrent responsiveness can be obtained by optimizing the organization of the SOS substrate, such as the impurity concentration of the silicon layer 2 and the quality of the insulator film. However, from the viewpoint of spatial resolving power, the thickness of the silicon layer 2 is preferably about 10 (μm) or less.

Here the upper limit of the film thickness X of the silicon layer can be defined by the wavelength λ used and the film quality of the silicon layer. In other words, since the silicon film thickness X should be smaller than the sum of the penetration depth and the diffusion length L of light, the following inequality, X<λ/(2πk)+L, needs to be satisfied. In the above inequality, k represents the extinction coefficient. Accordingly, the more the film quality of the silicon layer is improved, the larger the upper limit of the silicon film thickness becomes.

Preferably the lower limit of the film thickness X of the silicon layer is established so that the silicon layer can absorb all the incident light, in other words, desirably it is established so that the inequality, X>λ/(2πk), can be satisfied, it is not necessarily limited to this though.

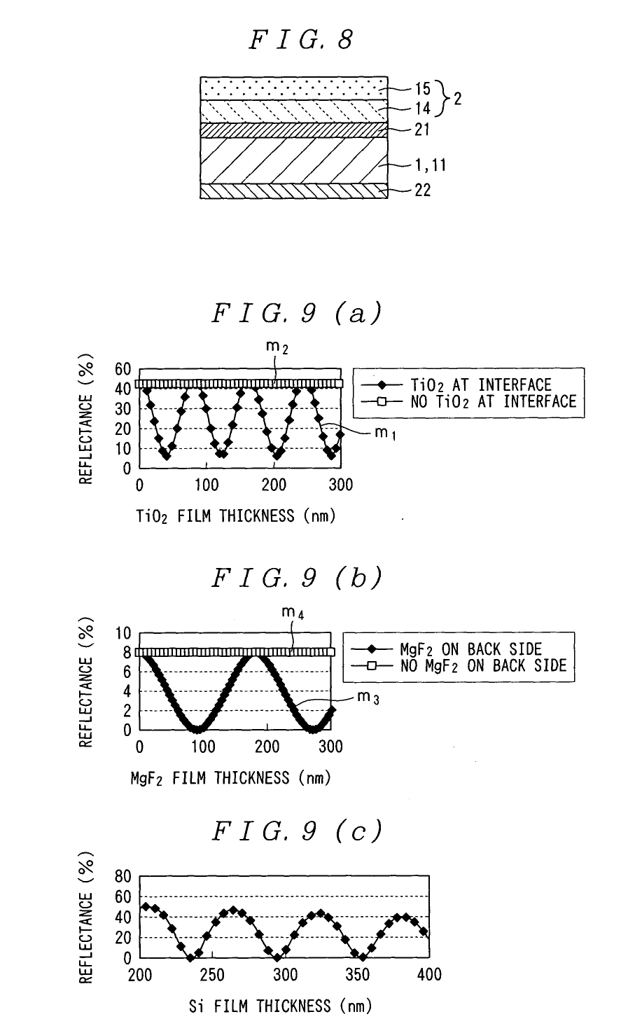

In the above described first embodiment, to inhibit incident light from being reflected by the surface of the light transmitting substrate land lost, desirably an antireflection film obtained by stacking optical thin films with different refractive indices, such as MgF₂, TiO₂ and SiO₂ films, is formed at least either on the surface of the light transmitting substrate 1 which is exposed to light or between the light transmitting substrate 1 (the sapphire substrate 11) and the single crystal silicon layer 2 (the seed silicon layer 14 and the second silicon layer 15), as shown in FIG. 8.

In this case, as the antireflection film 21 provided between the light transmitting substrate 1 and the single crystal silicon layer 2, those with a refractive index smaller than that of the single crystal silicon layer 2 and larger than that of the light transmitting insulator film can be used. And as the antireflection film 22 provided on the surface, which is to be illuminated with light, of the light transmitting substrate 1, those with a refractive index smaller than that of the light transmitting substrate 1 is desirably used.

For example, as for the silicon layer on the light transmitting substrate formed through the process shown in FIG. 3, an MgF₂ film can be applied as the antireflection film 22 on the surface of the sapphire substrate 11 which is exposed to light, and TiO₂ film can be applied as the antireflection film 21 between the sapphire substrate 11 and the seed silicon layer 14.

The antireflection film 22 provided on the surface of the light transmitting substrate, which is exposed to light, can be formed either before depositing the single crystal silicon layer 2 on the light transmitting substrate 1 or after forming the silicon layer on the light transmitting substrate. As for the antireflection film 21 provided between the light transmitting substrate 1 and the single crystal silicon layer 2, when preparing the silicon layer on the light transmitting substrate by epitaxy, it can be formed before depositing the single crystal silicon layer 2; and when preparing the silicon layer on the light transmitting substrate by bonding the light transmitting substrate 1 and the single crystal silicon layer 2 together, it can be formed on the surface of the silicon layer to which the light transmitting substrate 1 is to be bonded before the bonding.

The antireflection films 21 and 22 can be formed by the method such as sputtering, evaporation, or CVD.

FIG. 9(a) is a characteristic diagram showing the change in reflectance of the surface exposed to light with thickness of a TiO₂film when illuminating a laminated structure, obtained by providing the TiO₂ film as an antireflection film 21 between the sapphire substrate 11 and the single crystal silicon layer 2, with light having a wavelength of 500 (nm). In FIG. 9(a), the characteristic line m₁ shows the change in reflectance of the surface exposed to light with thickness of the TiO₂ film and the characteristic line m₂ shows the reflectance of the surface exposed to light when no antireflection film 21 is provided between the sapphire substrate 11 and the single crystal silicon layer 2.

As shown in FIG. 9(a), it is apparent that providing a TiO₂ film as an antireflection film 21 enables the reflectance of the surface exposed to light to be reduced, and moreover, optimizing the thickness of the TiO₂ film enables the reflectance to be kept to a minimum. Since a change in wavelength causes a change in characteristics of reflectance, optimizing the wavelength of the illumination light as well as the thickness of the TiO₂ film enables the reflectance to be reduced; accordingly, defining the thickness of the TiO₂ film so as to minimize the reflectance enables the reflection to be inhibited on the surface exposed to light, leading to realization of a molecule detecting sensor of higher sensitivity.

FIG. 9(b) is a characteristic diagram showing the change in reflectance with thickness of an MgF₂ film when providing an MgF₂ film as an antireflection film on the surface of the sapphire substrate 11 exposed to light. In FIG. 9(b), the characteristic line m₃ shows the change in reflectance with thickness of the MgF₂ film and the characteristic line m₄ shows the reflectance of the surface exposed to light when providing no MgF₂ film thereon. In this case also, it is apparent, as shown in FIG. 9(b), that providing an MgF₂ film enables the reflectance of the surface exposed to light to be reduced and setting the thickness of the MgF₂ film suitably according to the wavelength of light enables the reflectance to be kept almost zero.

FIG. 9(c) is a graph showing the change in reflectance of the surface exposed to light with thickness of the single crystal silicon layer 2 when illuminating a laminated structure made up of the sapphire substrate 11 (assuming that the film thickness is ∞) and the single crystal silicon layer 2 with light having a wavelength of 500 (nm).

As shown in the graph, it is apparent that optimizing the film thickness of the single crystal silicon layer 2 enables the reflection on the surface exposed to light to be approximately zero, provided that the wavelength is constant. Since a change in wavelength causes a change in characteristics of reflectance with the change in thickness of the single crystal silicon layer 2, optimizing the wavelength of the illumination light as well as the thickness of the single crystal silicon layer 2 enables the reflectance to be reduced to approximately zero; accordingly, defining the thickness of the single crystal silicon layer 2 so as to allow the reflectance to be approximately zero enables the reflection to be inhibited on the surface exposed to light, leading to realization of a molecule detecting sensor of higher sensitivity.

In this case, since electromagnetic-wave reflectance R can be calculated using the following equation (1), the film thickness and the wavelength can be set based on the equation (1).

R=|E₀^{-R}/E₀^{+R}|²=|S₂₁/S₁₁|² (1)

wherein E₀^{+R} represents the energy of the light entering a medium, E₀^{-R} the energy of the light reflected, and S₁₁, S₂₁ the matrix elements of the entire region obtained from the product of the characteristic matrices of the layers in the characteristic matrix method.

Thus, the reflectance on the surface exposed to light can also be reduced by optimizing the film thickness of the single crystal silicon layer 2, instead of providing the antireflection film described above.

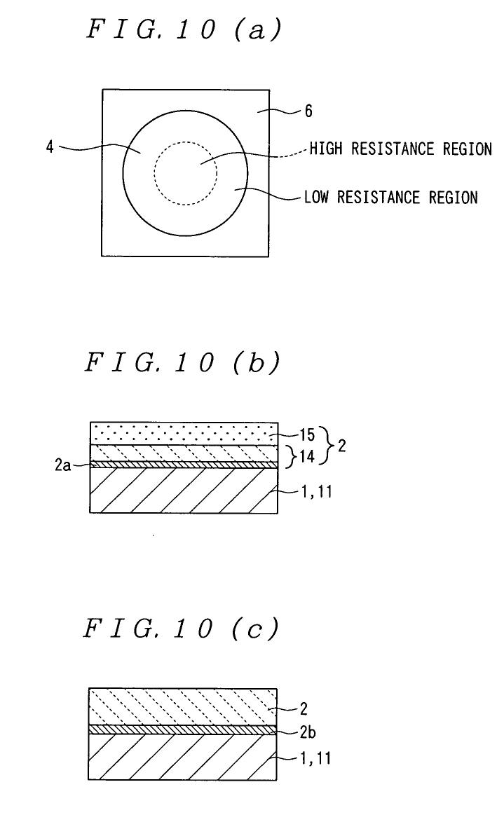

In the above embodiment 1, the thinner the silicon film, the higher the resistance of the same, and the more the region on the silicon away from the taking electrode 6, the higher the resistance in the region, causing current to be hard to draw in the region. And this causes changes in photocurrent characteristics. Specifically, current becomes harder to draw in the region closer to the center of the single crystal silicon layer 2, since the region closer to the center becomes more highly resistant, as shown in FIG. 10(a). When the resistance values of the single crystal silicon layer 2 have positional dependency, the true photocurrent characteristics according to the characteristics of the electrolyte 4 cannot be detected.

Thus, in order to avoid this problem, a heavily doped impurity layer 2a may be formed in the single crystal silicon layer 2 in the vicinity of the single crystal silicon layer 2-light transmitting substrate 1 interface, that is, in the seed silicon layer 14 in the vicinity of the seed silicon layer-sapphire substrate interface 11 as shown in FIG. 10(b), in the case of the SOS substrate of FIG. 3, by doping a high concentration of impurity of either n-type or p-type. Forming the heavily doped impurity layer 2a allows the single crystal silicon layer to be lower resistant, which in turn makes it possible to avoid the occurrence of positional dependency of resistance values in the single crystal silicon layer 2.

The heavily doped impurity layer 2a can be formed by ion implantation after the formation of the single crystal silicon layer 2 or by doping impurity at the same time as the epitaxial growth of the single crystal silicon layer 2.

That the ease of drawing current has positional dependency is problematic particularly in the molecule detecting sensors designed to detect changes in the state of an electrolyte by detecting changes in photocurrent characteristics; however, the positional dependency of the easy of drawing current can be avoided by doping a high concentration of impurity in the single crystal silicon layer and thereby the precision of the molecule detecting sensors can be improved.

In avoiding the positional dependency of the easy of drawing current, instead of the above described heavily doped impurity layer 2a, a transparent conductive film 2b may be formed between the light transmitting substrate 1, such as a sapphire substrate 11, and the single crystal silicon layer 2, as shown in FIG. 10(c). As the transparent conductive film 2b, for example, a film of ITO (indium tin oxide), zinc oxide (ZnO) with boron (B) or aluminium (Al) doped therein, or tin oxide with a halogen element such as fluorine doped therein is applicable.

The transparent conductive film 2b can be formed by the method such as sputtering, evaporation, or CVD. When preparing the SOI substrate by growing the single crystal silicon layer 2 by epitaxy, the transparent conductive film 2b can be formed on the light transmitting substrate 1 before forming the single crystal silicon layer 2. And when preparing the SOI substrate by bonding the single crystal silicon layer 2 and the light transmitting substrate 1 together, it can be formed on the surface of the light transmitting substrate 1 to which the single crystal silicon layer 2 is bonded before the bonding operation.

In the first embodiment described above, though a second silicon layer 15 is grown on the seed silicon layer 14 having been left after removing the silicon oxide layer 13, instead of providing the second silicon layer 15, an SOS substrate made up of the seed silicon layer 14 and a sapphire substrate 11 maybe applied. However, photocurrent characteristics can be much more improved when growing the second silicon layer 15 than when applying the SOS substrate, because the crystallinity of the seed silicon layer is insufficient and many crystal defects remains in the layer which can be the recombination center of optically pumped carriers.

In the first embodiment described above, after forming the first silicon layer 12, recrystallization may be performed for the layer through following the steps of: implanting ions in the layer, turn the deep portion of the same amorphous, and aneal treating the same. This can promote the progress of recrystallization in the silicon layer from the upper portion of relatively high crystallinity to the lower portion of relatively low crystallinity, which in turn improves the crystallinity of the entire silicon layer, decreases crystal defects of the same which can be the recombination center of optically pumped carriers, leading to improvement in photocurrent characteristics.

In the first embodiment described above, after forming the second silicon layer 15, a third silicon layer maybe grown by epitaxy on the second silicon layer 15 through following the same steps as described above, considering the second silicon layer 15 as the first silicon layer 12, that is, through following the steps of: oxidizing the second silicon layer 15; removing the silicon oxide formed in the oxidation step; and using the second silicon layer 15 left after the removing step as a seed silicon layer, and this treatment may be further repeated. This can improve the crystallinity of the silicon layer, decrease the crystal defects of the same, leading to improvement in photocurrent characteristics.

Next, a second embodiment of this invention will be described.

The second embodiment is the same as the first embodiment, except for the method for manufacturing the SOS.

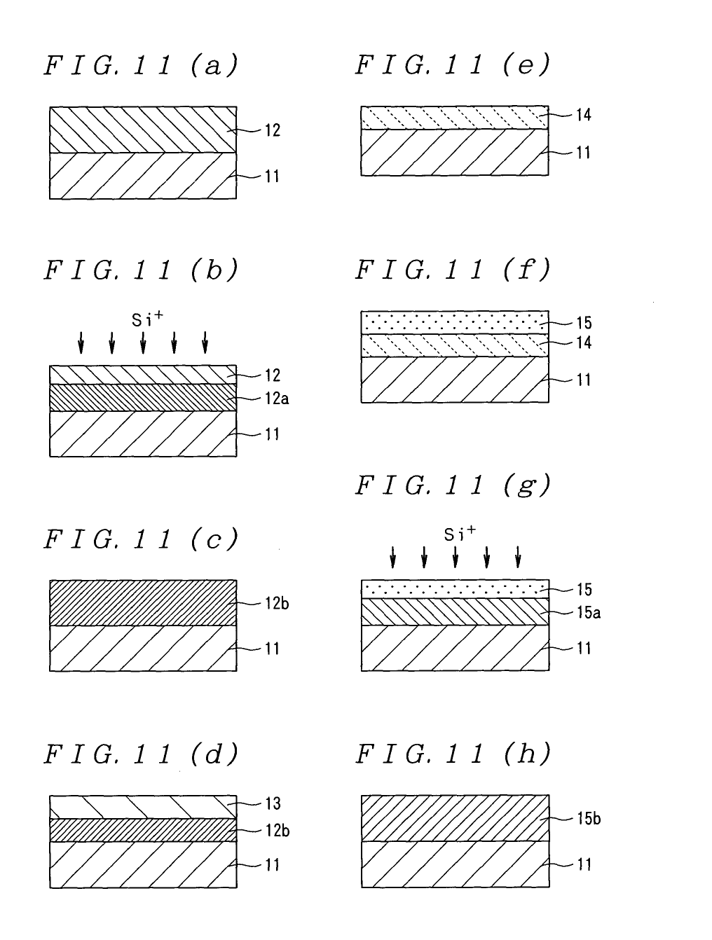

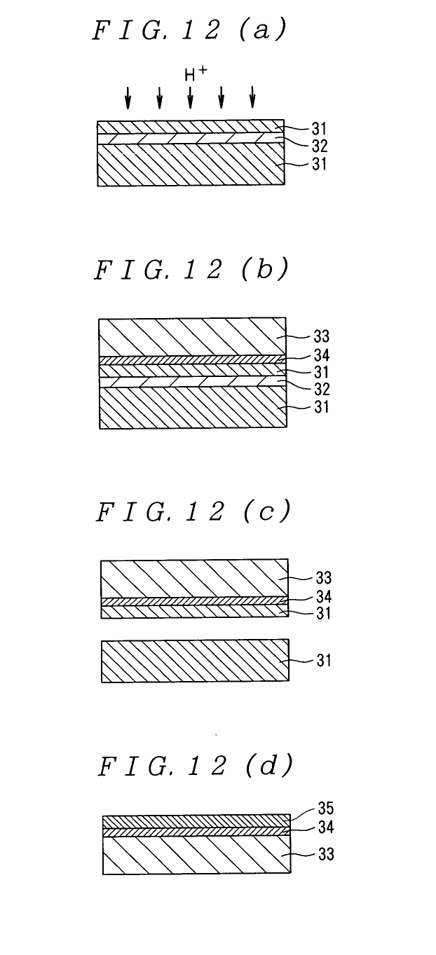

In the second embodiment, as shown in FIG. 11, a first silicon layer 12 is deposited on a R-plane sapphire substrate 11 (FIG. 11(a)).

Then, the first silicon layer 12 is turned amorphous (12a) in the vicinity of the first silicon layer 12-sapphire substrate 1 interface (FIG. 11(b)) by implanting silicon ions therein and recrystallized (12b) by heat treatment in the nitrogen atmosphere (FIG. 11(c)).

Subsequently, the sapphire substrate 11 is heat treated in the oxidizing atmosphere (FIG. 11(d)) to remove the silicon oxide layer 13 formed on the first silicon layer 12b which has been recrystallized (FIG. 11(e)).

Then a second silicon layer 15 is grown by epitaxy on a seed silicon layer 14, which is the first silicon layer after removing the silicon oxide layer 13, (FIG. 11(f)), the deep portion of the second silicon layer 15 is turned amorphous (15a) by implanting silicon ions in a laminated structure made up of the first silicon layer 14 and the second silicon layer 15 (FIG. 11(g)), and the deep portion having been turned amorphous is recrystallized (15b) by heat treatment in the nitrogen atmosphere (FIG. 11(h)).

The SOS substrate formed in the above manner also includes a silicon layer of high quality, just like that of the first embodiment, and provides the advantages equivalent to those of the first example.

In the second embodiment described above, a third silicon layer may also be grown by epitaxy on the epitaxially grown second silicon layer 15b after the step of recrystallizing the same. Alternatively, a third silicon layer may be grown by epitaxy on a part of the recrystallized second silicon layer 15b which is left after the steps of: oxidizing the recrystallized second silicon layer 15b by heat treatment in the oxidizing atmosphere; and removing the oxidized silicon layer by etching.

This can provide a silicon layer of high quality, that is, a silicon layer having higher crystallinity and fewer crystal defects.

Next, a third embodiment will be described.

In the third embodiment, an SOS substrate is formed using the method for forming a thin film on a stiffener, which is described in Japanese Patent Laid-Open No. 5-211128 Specification, and the detailed description will be omitted herein since they are disclosed in the specification.

As shown in FIGS. 12(a) to 12(d), first, hydrogen or rare gas ions are implanted in the surface of a single crystal silicon substrate 31 to form a layer 32 throughout which hydrogen or rare gas ions are distributed (FIG. 12(a)).

Second, the surface of the single crystal silicon substrate 31, in which the ions have been implanted, and a sapphire substrate 33 are bonded together with a transparent adhesive layer 34 consisting of an adhesive material inserted therebetween by applying pressure thereto (FIG. 12(b)).

Then, a single crystal silicon layer 35 is formed on the sapphire substrate 33 by heat treating the bonded substrates, to cause cleavage fracture in the single crystal silicon substrate 31 (FIG. 12(c)) due to the crystal reorientation therein and the pressing action of the fine gas bubbles formed therein through the ion implantation (FIG. 12(d)).

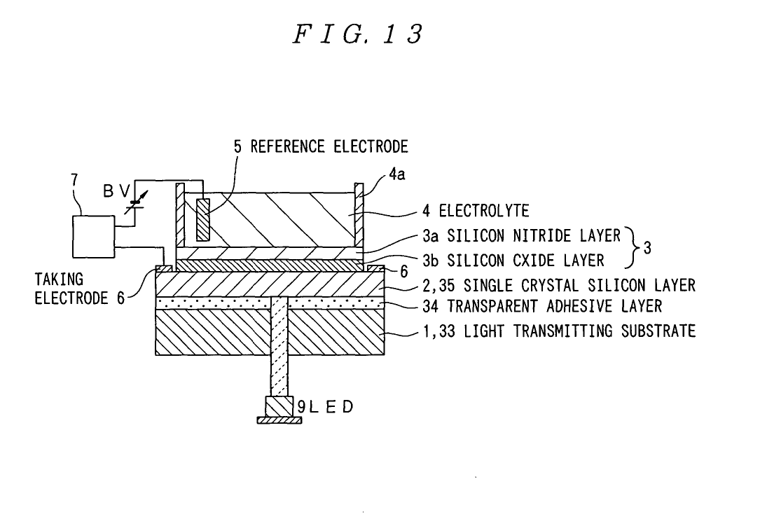

This can provide an SOS substrate with a single crystal silicon layer of excellent quality which consists of the sapphire substrate 33 and the single crystal silicon layer 35 formed thereon via the transparent adhesive layer 34 and including. Accordingly, a molecule detecting sensor manufactured using this SOS substrate, more specifically, manufactured in the steps of: forming a single crystal silicon layer 2 (35) on the sapphire substrate 33, as the light transmitting substrate 1, via the transparent adhesive 34; further forming an insulator layer 3 by stacking a silicon oxide layer 3b and a silicon nitride layer 3a in this order on the single crystal silicon layer 2 (35); and forming an electrolyte 4 on the insulator layer 3, as shown in FIG. 13, provides advantages equivalent to those of the first embodiment.

Next, a fourth embodiment will be described.

In the fourth embodiment, an SOS substrate is formed using the “bonded SOI” described in Japanese Patent Laid-Open No. 7-235651 Specification, and the detailed description will be omitted herein since they are disclosed in the specification.

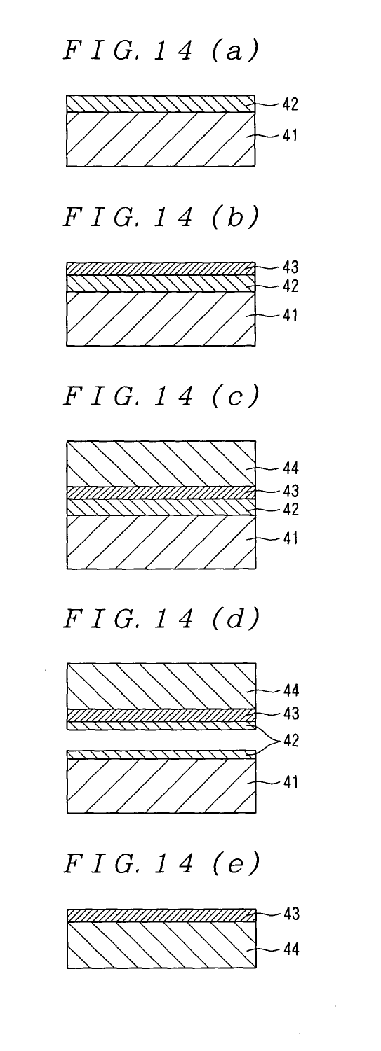

As shown in FIGS. 14(a) to 14(e), first, a porous silicon layer 42 is formed on the surface of a single crystal silicon substrate 41 by anodizing the same (FIG. 14(a)). Second, a nonporous single crystal silicon layer 43 is grown by epitaxy on the porous silicon layer 42 (FIG. 14(b)).

Then, for example, a sapphire substrate 44 as a light transmitting substrate is bonded to the surface of the nonporous single crystal silicon layer 43. The bonding is performed after washing both substrates in a mixed solution of hydrochloric acid and hydrogen peroxide by applying pressure thereto (FIG. 14(c)). Alternatively, the substrates may be bonded together with a transparent adhesive layer 34 inserted therebetween by applying pressure thereto, as shown in FIG. 12(b).

Then, the single crystal silicon substrate 41 and the porous silicon layer 42 are removed while leaving the epitaxially grown nonporous single crystal silicon layer 43. In this removing operation, the single crystal silicon substrate 41 is removed in two steps: grinding and etching and the porous silicon layer 42 is removed by selective etching after completely removing the single crystal silicon substrate 41 (FIG. 14(d)).

Thus, an SOS substrate with the nonporous single crystal silicon layer 43 formed on the sapphire substrate 44 can be obtained (FIG. 14(e)).

The SOS substrate formed in this manner can include a single crystal layer of excellent quality, and the use of the SOS substrate can provide advantages equivalent to those of the SOS of the embodiment 1.

While the embodiments 3 and 4 have been described taking an example where the sapphire substrate 11 is used as the light transmitting substrate 1, the light transmitting substrate is not limited to the sapphire substrate 11, but a single crystal oxide substrate, a glass substrate consisting of an amorphous material such as SiO₂, or a plastic substrate of, for example, polycarbonate is also applicable.

Further, in the embodiments 3 and 4, before the step of bonding a sapphire substrate 44 to the surface of the nonporous single crystal silicon layer 43 which is shown in FIG. 14(c), a transparent conductive film such as an ITO film may be formed on the surface of the nonporous single crystal silicon layer 43, and the nonporous single crystal silicon layer 43 with a transparent conductive film can be bonded to a carrier substrate, in this case, the sapphire substrate 44.

Further, before the bonding operation, a heavily doped region may be formed in the vicinity of the single crystal silicon surface, to which a sapphire substrate is to be bonded, by heat treating the surface in the dopant gas atmosphere or adding a dopant to the surface, and the single crystal silicon layer with a heavily doped region can be bonded to a carrier substrate.

In the second and fourth embodiments, too, doping a high concentration of impurity in the single crystal silicon layer in the vicinity of the single crystal silicon layer-sapphire substrate interface, or forming a transparent conductive film between the sapphire substrate and the single crystal silicon layer makes it possible to avoid the occurrence of positional dependency in the ease of drawing current, leading to improvement in photocurrent characteristics.

Further, an antireflection film may be provided at least either between the single crystal silicon layer and the sapphire substrate or on the sapphire substrate surface exposed to light. Providing an anti reflection film in such a manner enables the improvement of photocurrent characteristics.

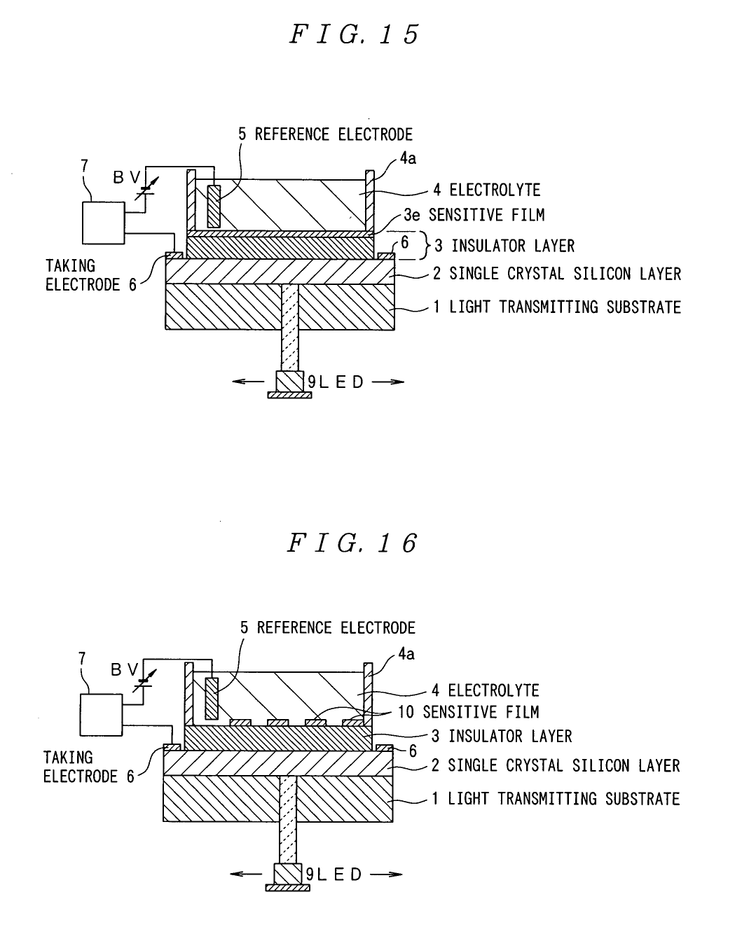

In each of the above described embodiments, as shown in FIG. 15 a wide variety of substances can be detected by forming a sensitive film 3e, of which charge changes according to the electrolyte 4 as an object of measurement, on the insulator layer 3 and replacing it according to the object of measurement. For example, if a silicon nitride film or a lipid film or a platinum thin film is formed as a sensitive film 3e, the sensor can be used as a pH detecting sensor or a taste sensor or a gas sensor; thus, forming an arbitrary film as a sensitive film 3e allows a wide variety of sensors to be formed.

Further, the sensors may be used as an image sensor if they are allowed to scan light beams. In addition, as shown in FIG. 16, the sensors may also be used as a multi-array sensor if a plurality of individual sensors 10 consisting of different types of sensitive films are arrayed on the insulator layer 3 and an electrolyte as an object of measurement is allowed to come in contact with the respective individual sensors 10. In either case, an image sensor or a multi-array sensor can be constructed which has high resolution and high sensitivity compared to those currently in use. At the time of using the sensors as a multi-array sensor, the multi-array sensor may be constructed so that it can scan light beams or so that each sensitive film is provided with a light emitting diode (LED).

According to the molecule detecting sensors of this invention, the area of a sensitive film can be reduced because they have higher resolution and higher sensitivity, and they can be suitably used as a multi-array sensor because the amount of a sample required for one examining item can be reduced, in other words, more items can be examined with a smaller amount of sample.

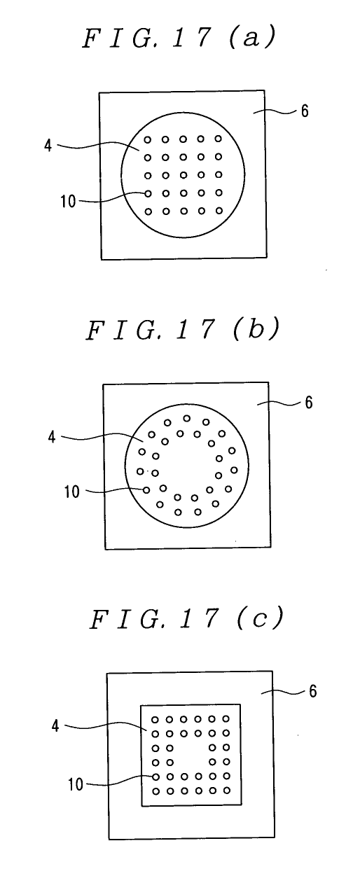

At the time of using the sensors as a multi-array sensor, if a plurality of individual sensors 10 consisting of different sensitive films are arranged in rows and columns as shown in FIG. 17(a), the multi-array sensor is affected by the positional dependency of the resistance values in the single crystal silicon layer 2 since the current is harder to draw in the single crystal silicon layer 2 at a position which is more distant from a taking electrode 6. In this case, too, the problem can be avoided by implanting a high concentration of impurity in the same manner as above; however, as shown in FIG. 17(b) if the opening of the taking electrode 6 is circular, a plurality of individual sensors 10 maybe arranged on a circle concentric with the opening, or on 2 or 3 circles concentric with the opening.

At the time of arranging a plurality of individual sensors 10 on a plurality of concentric circles, the individual sensors 10 on a circle are the same distance from the taking electrode 6 and the resistance values at the positions of the individual sensors 10 on a circle are the same; accordingly, analyses can be done using the photocurrent values obtained in the steps of: detecting photocurrent induced by the light illumination performed on the respective individual sensors 10 on each circle under the same conditions; by using the photocurrent value of the individual sensors 10 on, for example, the outermost circle as the basis, calculating amplification ratios used to allow the photocurrent values detected from the individual sensors on the concentric circles other than the outermost circle to correspond to those detected from the sensors on the outermost circle; and amplifying the photocurrent values detected from the individual sensors 10 on the concentric circles other than the outermost circle on the basis of the amplification ratio calculated in the above step.

In other words, the effect of the positional dependency of the resistance values in the single crystal silicon layer 2 on photocurrent induced can be removed by adjusting the photocurrent values detected; accordingly, a multi-array sensor can be obtained which is not affected by the positional dependency of the resistance values in the single crystal silicon layer 2. In this case, since what to do is just adjusting photocurrent values detected, the procedure for avoiding the effect of the positional dependency of resistance values is easier than that used when implanting a high concentration of impurity.

The individual sensors 10 are not necessarily arranged to take the form of concentric circles; for example, as shown in FIG. 17(c), if the taking electrode 6 is formed so that its opening is in the form of a square, the individual sensors 10 can be arranged in the form of squares concentric with the square opening of the electrode 6, or it may be possible to arrange them to take the form of triangles concentric with the triangular opening of the electrode. The most important thing is to arrange the individual sensors 10 at positions which are the same distant from the taking electrode 6.

In these cases, the individual sensors 10 can be easily arranged at positions the same distant from the taking electrode 6 through following the steps of: for example, removing the insulator layer 3 while allowing the part corresponding to the circular electrolyte region, on which an electrolyte is placed, to be left at the center portion of the laminated structure made up of the light transmitting substrate 1, the semiconductor layer 2 and the insulator layer 3; forming a taking electrode 6 around the periphery of the electrolyte region on the semiconductor layer 2; placing an electrolyte 4 in the opening of the taking electrode 6; arranging sensitive films on the periphery of figures on the electrolyte 4 which are similar to and concentric with the opening of the taking electrode 6, that is, on the periphery of circles concentric with the opening of the electrode 6 if the opening is circular.

For the multi-array sensor, if sensitive films are applied which undergo changes in physical properties, which are detectable with an image sensor, due to the mutual reaction with, for example, proteins, substrates of enzymes and nucleic acids, the physical properties of substances mutually reacting with the sensitive films can be detected.

For example, if a specific enzyme having been fixed is applied as a sensitive film, the concentration of its substrate contained in a measuring sample can be determined. If oxidase, such as cytochrome P450, which is involved in the oxidation of various substances and has many subtypes, is selected as a group of enzymes used in the multi-array and a pattern analysis is performed comprehensively for the data obtained by detecting the signals from the enzyme spots one by one, the multi-array sensor can be used as a sensor for identifying the substances in the sample. This enables the simultaneous measurement of multiple examining items and can be applied to measurements of biochemical items in blood examination.

If a nucleic acid having a sequence complementary to the sequence of the nucleic acid as a detecting object is fixed, the sensor can be used for DNA chips applicable to the screening of pharmaceuticals, gene diagnosis chips, chips for analyzing the toxicity occurrence probability of drugs using Single Nucleic Acid Polymorphism s (SNPs) diagnosis or drug sensitivity, infectious disease diagnosis chips, nucleic acid-fixed resistant bacteria detection chips using a resistant bacteria gene panel, etc.

As a nucleic acid fixed as a sensitive film, any one of DNA, RNA, or other nucleic acids similar in structure to the nucleic acid as a measuring object is applicable, as long as it hybridizes to the nucleic acid as a measuring object.

A method may also be used in which an antihapten antibody having been fixed as a sensitive film is indirectly bound to a nucleic acid labeled with hapten. To detect nucleic acid with higher sensitivity, a charged intercalator molecule to which, for example, ferrocene is bound, or a mediator, or the combination thereof may be used.

A method may also be used in which detection is made using a detecting nucleic acid directly or indirectly labeled with oxidase.

The above described sensitive films are formed in an array at the discontinuous sites of the activated surface of the insulator layer. The other active sites may be blocked, if necessary. The sensitive films can be formed using, for example, a silane coupling agent.

The term “silane coupling agent” herein used means an organosilicon compound having an organic functional group, such as vinyl-, epoxy-, amino- or mercapt-group, which has an affinity for organic materials and a hydrolytic group, such as methoxy- or ethoxy-group, which has an affinity for inorganic materials in its molecule.

The hydrolytic group in a silane coupling agent combines with the hydroxyl group exposed on the surface of the insulator layer and the organic functional group in the same combines with a protein, enzyme or nucleic acid.

The protein, enzyme or nucleic acid may be bound to the insulator layer via a linker.

Any silane coupling agents are applicable to this invention as long as they correspond to the above described definition.

The silane coupling agents applicable to this invention include, for example, 3-aminopropyl triethoxy silane, 3-aminopropyl trimethoxy silane, 3-aminopropyl diethoxymethyl silane, 3-(2-aminoethyl aminopropyl) trymethoxy silane, 3-(2-aminoethyl aminopropyl) dimethoxymethyl silane, 3-mercaptpropyl trimethoxy silane, and dimethoxy-3-mercaptpropyl methyl silane, and they can be used independently or in combination.

To provide a highly efficient coupling reaction with a silane coupling agent, a bifunctional reagent, such as heterobifunctional linker, can sometimes be used.

The bifunctional reagents used are not necessarily limited as long as they allow the intended protein, enzyme or nucleic acid to be immobilized on a covalent bond.

The bifunctional reagents applicable to this invention include, for example, glutaraldehyde, periodic acid, N, N-o-phenylene dimaleimide, N-succinimidyl-4-(N-maleimide methyl) cyclohexane-1-carboxylate, N-succinimidyl maleimidbutyric acid, N-succinimidyl-4-maleimidbutyric aid, N-succinimidyl-6-maleimidhexanoic acid, N-sulfosuccinimidyl-4-maleimidmethylcyclohexane-1-carboxylic acid, N-sulfosuccinimidyl-3-maleimidbenzoic acid, N-(4-maleimidebutyloxy) sulfosuccinimide sodium salt, N-(6-maleimidecaproyloxy) sulfosuccinimide sodium salt, N-(8-maleimidecapryloxy) sulfosuccinimide sodium salt, N-(11-maleimideundecanoyloxy) sulfosuccinimide sodium salt, and N[2-(1-piperazinyl)ethyl]maleimide dihydrochloric acid, etc., and they can be used independently or in combination.

Further, to provide a coupling reaction, a photoactive crosslinking agent may be used.

For example, a photoactive crosslinking agent, such as N-hydroxysuccinimid of p-aminobenzophenone or p-azidobenzoic acid, is reacted on the surface of the insulator layer having been treated with a silane coupling agent in a darkroom. Then, the target protein, enzyme or nucleic acid is spotted in a desired manner, irradiated with ultraviolet rays for a short period of time or irradiated with visible rays for a longer period of time to accomplish covalent attachment. The region on the surface of the insulator layer left unreacted is blocked using a blocker similar to the molecules described above in the presence of ultra violet rays or visible rays. Or the target protein, enzyme or nucleic acid can be fixed using a photopolymer such as a copolymer of dimethylacrylamide and cinnamoyloxyethyl methacrylate and then covalently attached on the silicon surface using a photopolymer such as polymetaazidostyrene.

With the methods described above, proteins, enzymes or nucleic acids, etc., can be fixed on sensitive films as multi-array sensors of a chemical image sensor.

The molecules of the fixed proteins, enzymes or nucleic acids, etc., maybe stabilized, and one example of the methods for stabilizing the molecules is to incubate them in a solution of sugar such as trehalose for a short period of time (e.g. 1 hour) and dry at 37 (° C.) for 16 hours. Then the stabilized sensitive films are hermetically sealed and preserved in a small foil bag with a desiccant. The fixed molecules are stable for 6 to 12 months or more, and if they are preserved at 2 to 8 (° C.), they are stable for 2 years or more.

Next, another molecule detecting sensor to which this invention is applied will be described.

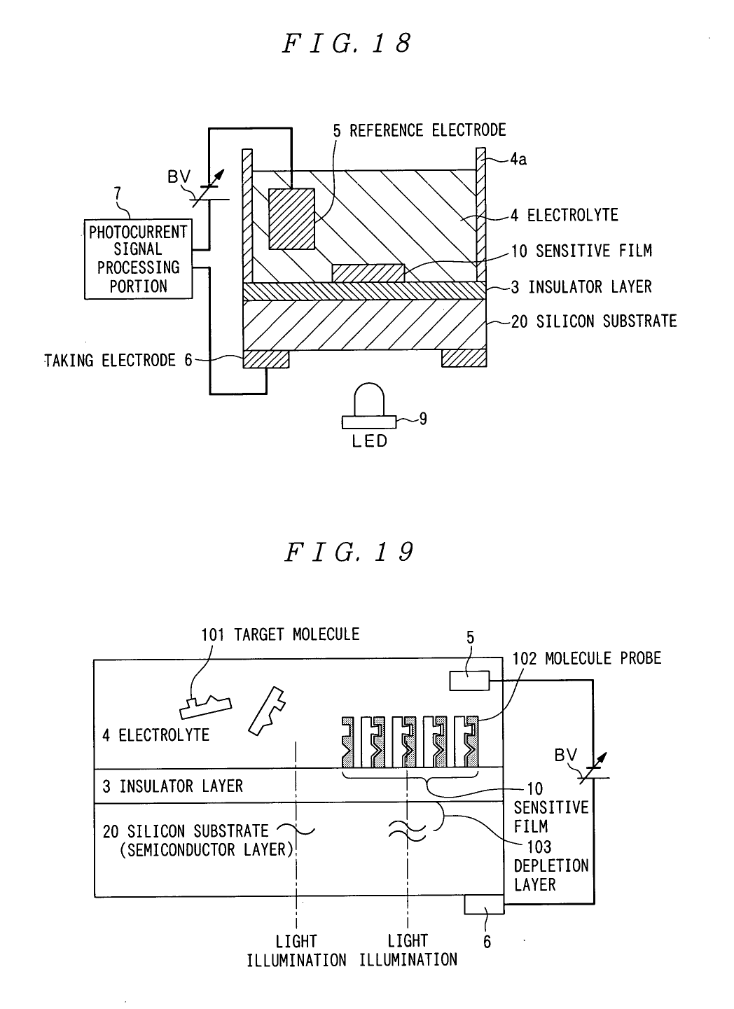

This molecule detecting sensor is a molecule detecting sensor designed to detect a specific molecule by providing a sensitive film 10, which causes a change in charge with respect to the specific molecule, on an insulator layer 3. As shown in FIG. 18, the sensitive film 10 is placed on the insulator layer 3 superposed on a silicon substrate 20.

And on the sensitive film 10 is formed a molecule probe 102 which combines with a target molecule (a specific molecule) 101, as a detecting object, described later in FIG. 19.

Specifically, when the target molecule 101 combines with the molecule probe 102, a change is caused in width of a depletion layer 103 having been created in the semiconductor layer in the vicinity of the insulator layer 3-semiconducotr layer interface, which in turn causes a change in quantity of the photocurrent induced by the illumination of light using a light emitting diode 9; and, the molecule detecting sensor is designed to detect a specific chemical quantity/substance in an electrolyte by detecting the change in quantity of the photocurrent induced.

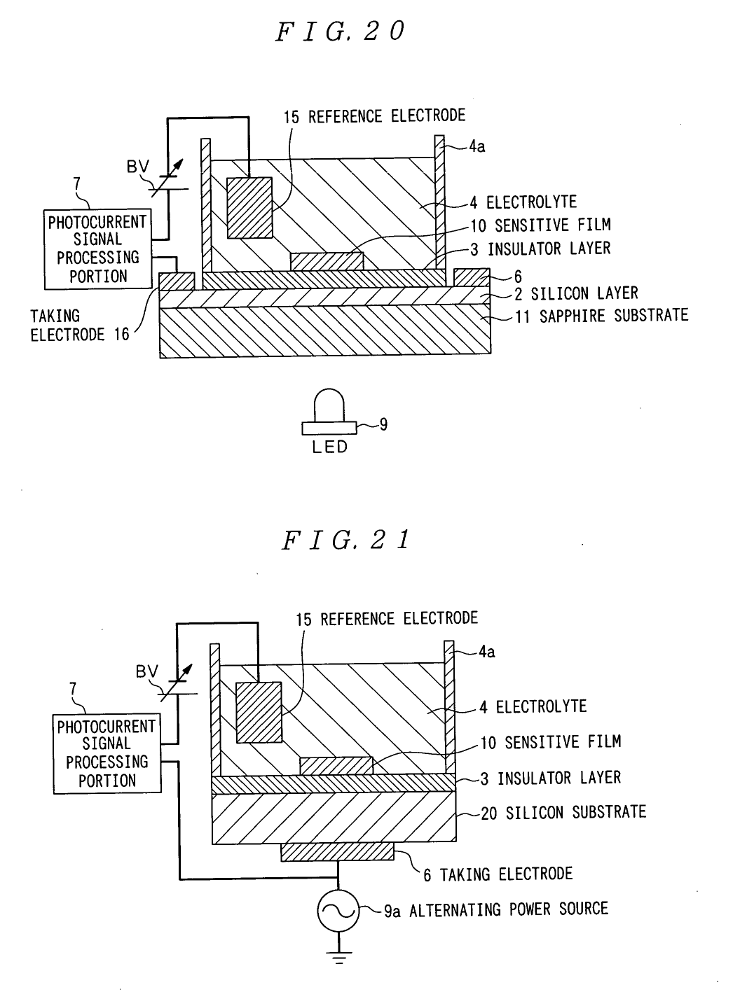

As the silicon substrate 20, desirably a single crystal silicon substrate having a polished and smooth surface is used. However, instead of the silicon substrate 20, an insulator substrate, as a carrier substrate, with a semiconductor thin film placed thereon can also be used. For example, an SOI substrate made up of an insulator substrate and a single crystal silicon thin film layer formed thereon or an SOS substrate made up of an insulator substrate consisting of sapphire 11 and a silicon layer formed thereon, as shown in FIG. 20, can also be used. When using such an SOI substrate, the semiconductor layer (silicon layer 2) can be made thin compare to when using no carrier substrate.

Specifically, when using a modulated light as means for stimulating a semiconductor substrate or a semiconductor layer, the information obtained depends on the area exposed to light. Since the beam radius of coherent light such as laser beam depends on its wavelength, in order to improve the resolution of a LAPS sensor when it is used in a microscopic application or the array density of the same when it is used in an array sensor application, it is necessary to decrease the beam radius of light by decreasing the wavelength of light.

In a single crystal silicon substrate, however, it is difficult to decrease the wavelength of light because when using a blue light source with a wavelength of 500 (nm) or less, the carriers having been generated are diffused and deactivated before they reach the semiconductor-insulator interface, due to its large thickness, and thereby no photocurrent is induced. In a bulk silicon substrate, when light illumination is performed from its back, carriers are generated in the neutral region to which electric field is not applied and only those having reached the depletion layer due to their diffusion induce photocurrent.

On the other hand, at the time of using an SOI substrate, it is preferable from the viewpoint of sensitivity that light illumination is performed so that optically pumped carriers are generated in the depletion layer and its vicinities. Specifically, in an SOI substrate made up of a light transmitting substrate and a silicon layer, of which thickness is several (μm), formed thereon, carriers are generated even in the depletion layer and almost all the carriers having been generated in the depletion layer induce photocurrent when applying an electric field thereto. This is effective in increasing the sensitivity of the molecule detecting sensor, in which the SOI substrate is superior to the bulk silicon substrate.

The insulator layer 3 is a silicon dioxide layer formed by subjecting the surface of the silicon substrate 20 to thermal oxidation.

The film thickness of the insulator layer 3 can be on the order of 500 (nm) or less, and from the viewpoint of sensitivity, desirably it is on the order of 100 (nm) or less. Since moisture permeates the silicon dioxide when it is brought into contact with an electrolyte 4, desirably a thin film consisting of any one of silicon nitride, aluminium oxide and tantlum oxide is formed on the silicon dioxide layer. Alternatively, instead of forming the silicon dioxide layer, that is, the insulator layer 3 on the silicon substrate 20, such a dielectric layer may be formed directly on the silicon substrate 20. In this case, the film thickness of the dielectric layer can be on the order of 500 (nm) or less, and from the viewpoint of sensitivity, desirably it is on the order of 100 (nm) or less.

The above described sensitive film 10 is formed on the insulator layer 3, which comes in contact with the electrode 4, in such a manner as to include the molecule probe 102 having a sequence of gene complementary to one that is to be determined. As the molecule probe 102, any probe can be used as long as it can hybridize to DNA or RNA extracted from a biological sample, or modified by fragmentation etc., or synthesized in vitro, or to DNA, RNA or PNA etc., chemically synthesized.

The molecule probe 102 is formed into a sensitive film 10 on the insulator layer 3 which comes in contact with the electrode 4, and desirably it is fixed so that it will not flow out, because it requires different types of washing during the process of hybridization.

The fixation may be performed in the following manner.

A chloroform solution containing 0.2% 3-aminopropyl ethoxy silane is prepared in a flask, a substrate with an insulator layer is immersed in the solution, and the solution in the flask is replaced by argon gas or nitrogen gas. The substrate is left standing for about 20 hours while keeping the inside of the flask at 48 (° C.). Then the substrate is taken out and dried in nitrogen gas.

Then, N-succinimidyl-6-maleimidhexanoic acid, as a bifunctional reagent, is dissolved in DMSO to a concentration of 20 (mg/ml), the substrate having undergone silane coupling reaction is immersed in the solution and left standing for about 4 hours at 35 (° C.) . After the reaction, the substrate is washed in pure water.

DNA with thiol groups having been introduced to its terminals is dissolved with TE buffer (10 (mmol/l) Tris-HCL, 1 (mmol/l) EDTA, pH 8.0) so that its concentration is changed from 1 (μmol/ml) to 1 (pmol/ml).

An appropriate amount of DNA solution is placed on the above substrate and left standing for about 20 hours at 25 (° C.) in such a manner as not to allow the DNA solution to evaporate. Then 1 (mmol/l) 2-mercaptethanol is placed on the substrate and left standing for about 4 hours at 25 (° C.). The substrate is then washed in TE buffer to give a DNA-fixed substrate.

Alternatively, the fixation may be performed in the following manner.

Thin films of Ti/Au with a thickness of 25 (nm)/50 (nm), respectively, are formed in this order on the substrate at portions where DNA is fixed by the EB evaporation method.

DNA with a thiol group having been introduced to its terminal is dissolved with TE buffer (10 (mmol/l) Tris-HCl, 1 (mmol/l) EDTA, pH 8.0) so that its concentration is changed from 1 (μmol/ml) to 1 (pmol/ml).

An appropriate amount of DNA solution is placed on the above substrate and left standing for about 20 hours at 25 (° C.) in such a manner as not to allow the DNA solution to evaporate. Then 1 (mmol/l) 2-mercaptethanol is placed on the substrate and left standing for about 4 hours at 25 (° C.). The substrate is then washed in TE buffer to give a DNA-fixed substrate.

The fixing methods are not limited to the above described ones, but other known fixing methods, such as a method to use a functional group exposed on the surface of the insulator layer 3 to bond DNA and the substrate together or a method to fix gold on the surface of the insulator layer 3 and use the gold to bond substances having undergone various types of modification thereto, can also be used.

The gene having been extracted from a sample is hybridized with the sensitive film 10 as it is, or after modified by fragmentation, or after amplified. During the hybridization, the temperature condition of the sensitive film 10 as well as the electrolyte 4 need to be controlled, and such control may be performed by keeping the entire substrate, including the electrode, in a thermostatic bath or by using a Peltier device.

The gene not combined with the sensitive film 10 is removed by washing, and the washing can be performed by any one of the methods in common use. Further, DNA not combined with the sensitive film 10 is removed utilizing the charge on gene.

The hybridization is not necessarily performed in the presence of an intercalator; however, to increase the sensitivity, it may be performed in the presence of an intercalator, as a bonding molecule, or a protein such as an antibody which can combine with a nucleic acid or the derivative thereof, or in the presence of a nucleic acid or the derivative thereof, other than the molecule probes 102, which hybridizes to the nucleic acid or the derivative thereof.

Preferably the above bonding molecules are modified with urease or ferrocene. The reason is that the LAPS method is suitable for detecting a change in pH value. Specifically, if a double-stranded DNA bonding intercalator modified with, for example, urease is reacted after hybridization, only the portions where the double-stranded DNA exists are allowed to have the intercalator, and if the electrolyte solution has urea added thereto, the urease having been bound to the intercalator decomposes the urea in the solution, which can be detected as a change in pH.

However, the classical intercalators conventionally used combine with not only double-stranded DNA, but also single-stranded DNA, which makes it difficult to discriminate between single-stranded DNA and double-stranded DNA. In recent years, however, intercalators which combine specifically with double-stranded DNA have been developed, and herein these intercalators are referred to as double-stranded DNA bonding intercalator. As the above described intercalator, a double-stranded DNA bonding intercalator is preferably used. However, when the specific molecules are sufficiently large compared to the molecule probes 102, molecules which can combine with the specific molecules, for example, molecules which can combine with single-stranded DNA (including intercalators) may also be used; in this case, the molecule detecting sensitivity can be improved.

In such a state, an arbitrary portion of the silicon substrate 20 is illuminated with a beam of modulated light having a wavelength determined based on the film thickness of the silicon substrate 20. And bias voltage BV is applied to a reference electrode (a second electrode) 5 as well as a taking electrode (a first electrode) 6, and photocurrent thereby induced is detected at a photocurrent signal processing portion (electric characteristics detecting means) 7.

The bias voltage BV is set as follows. The bias voltage BV, which changes from an inversion state to a storage state depending on the polarity of semiconductor caused when changing the bias voltage BV from positive to negative, or vice versa, induces photocurrent depending on the surface state of the insulator layer 3.

In this case, if photocurrent is plotted in ordinate and bias voltage in abscissa, a kind of voltamograph can be obtained. In the voltamograph obtained when a DNA is hybridized at the portion where a single-strand DNA is fixed, the formation of a double-stranded DNA is observed as a change in bias voltage BV at which photocurrent rises.

Accordingly, when fixing bias voltage BV at an arbitrarily value between the value which allows photocurrent to rise and the value which allows the photocurrent to be saturated, if the DNA hybridization is carried out, the existence of the target DNA can be detected as a change in photocurrent value.

The existence of the target DNA can also be detected by detecting the difference in induced photocurrent value between the portion where a DNA probe is fixed and the reference portion, that is, the portion where no DNA probe is fixed.

Further, since the bias voltage can be changed arbitrarily, setting the polarity of the silicon substrate 20 to positive with respect to negatively charged DNA allows hybridization reaction to proceed rapidly, due to the Coulomb attraction between the silicon substrate 20 and DNA. Then, setting the polarity of the silicon substrate 20 to negative allows incomplete hybridization to untie, if there exists incomplete DNA-DNA hybridization, due to the Coulomb repulsion. This can improve accuracy of detection.

The molecule detecting sensor can be provided with one or more sensitive film 10 described above. If only one sensitive film 10 is provided, the sensor can be used as a sensor for detecting only one molecule, and if a plurality of sensitive films are provided, it can be used as a multi-array sensor.

When providing a plurality of sensitive films 10, they are arranged so that they are not affected by the positional dependency of the resistance values of the silicon layer, as described above, and the silicon substrate 20 may be illuminated with light from, for example, a light emitting diode 9, preferably a laser, or a near-field light in such a manner as to allow the light to scan the silicon substrate 20 or to move the silicon substrate 20 relative to the light emitting diode 9 or the laser. A light emitting diode may be provided for each sensitive film 10 to scan the silicon substrate 20 or laser light may be allowed to scan the same with the aid of a mirror etc.

Although providing a plurality of sensitive films 10 which combine with respective different specific molecules allows the molecule detecting sensor to be used as a multi-array sensor, a plurality of sensitive films 10 all of which combine with the same specific molecule may also be provided. This can improve accuracy of detection, when the specific molecule is sufficiently large, compared to the molecule probe 102.

While the molecule detecting sensor for detecting a specific molecule has been described in terms of using light illumination to induce photocurrent, this invention is not intended to be limited to this; and photocurrent maybe induced using, for example, a modified electric signal.

FIG. 21 shows a molecule detecting sensor which uses a modified electric signal as means for stimulating a silicon substrate 20 and in which an alternating power source 9a for applying an electric signal is provided instead of a light emitting diode 9, the alternating power source 9a being connected to a taking electrode 6.

The molecule detecting sensor is designed to detect the electrochemical state at the electrolyte-insulator layer interface by applying a modulated electric signal from the alternating power source 9a and bias voltage BV superposed on each other between a reference electrode 5 and the taking electrode 6 and measuring alternating current which depends on the spread of a depletion layer.

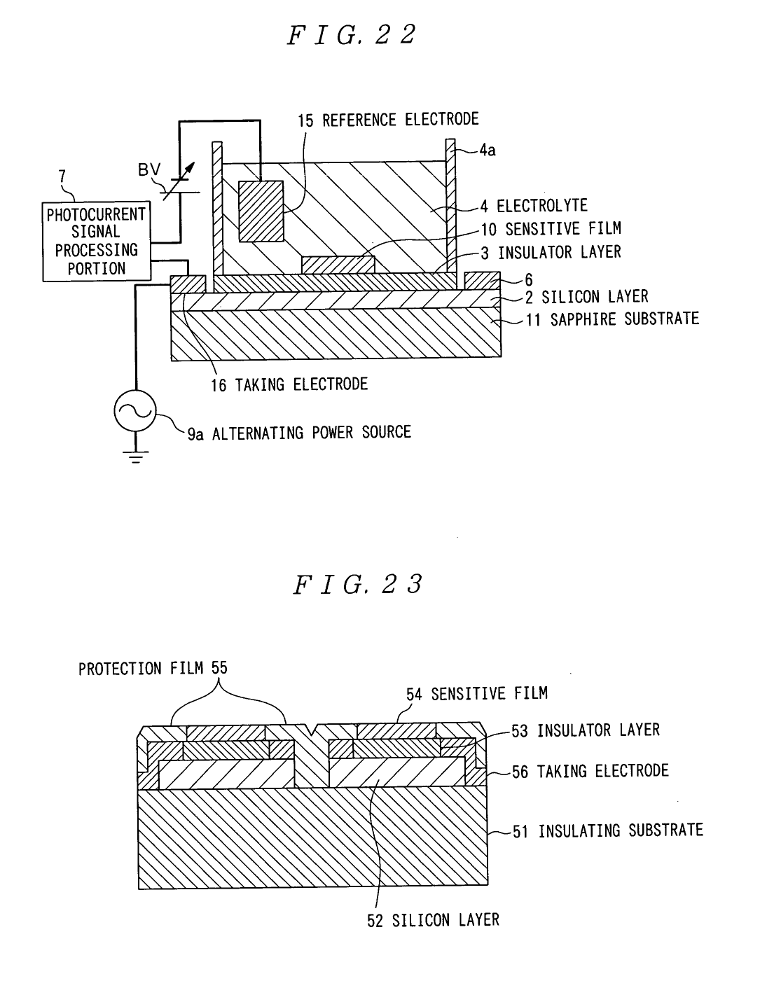

In this case, too, instead of the silicon substrate 20, an insulator substrate having a semiconductor thin film placed thereon maybe used, and an SOI substrate made up of an insulator substrate and a single crystal silicon thin film layer formed thereon or an SOS substrate made up of a sapphire substrate 11 as an insulator substrate and a silicon layer 2 superposed thereon, as shown in FIG. 22, can also be used.

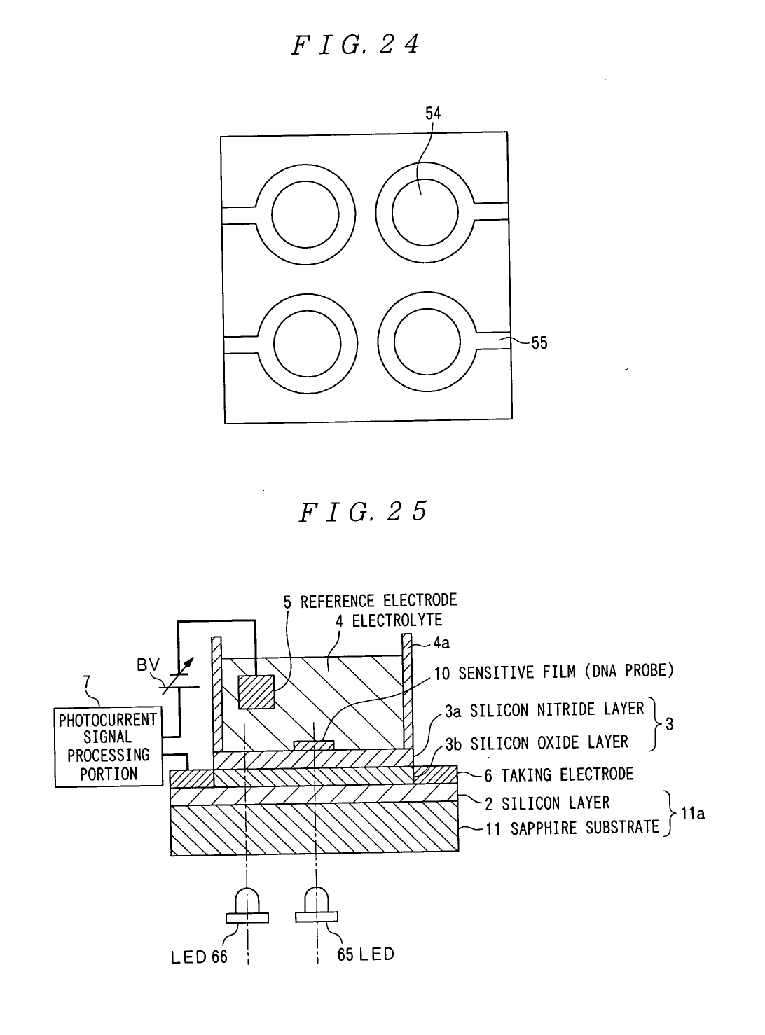

The molecule detecting sensor can be used as a multi-array sensor if a plurality of sensitive films 10 constructed so as to include different kinds of molecule probes are arranged on the insulator layer 3. In this case, to drive each array, which includes a molecule probe, independently, desirably the arrays are isolated from each other, as shown in the cross-sectional view of FIG. 23 and the top view of FIG. 24. The multi-array sensor can be formed using an SOI substrate as a substrate and performing isolation thereon. And such a SOI substrate may have a structure in which an insulator layer is buried in a semiconductor layer, just like SIMOX substrate, and the most important thing is the substrate can undergo isolation.

However, considering that the adjacent arrays need to be electrically completely isolated, desirably an SOI substrate is used as a semiconductor substrate, as shown in FIG. 23.

In FIG. 23, reference numeral 51 denotes an insulating substrate, reference numeral 52 a silicon layer, reference numeral 53 an insulator layer, reference numeral 54 a sensitive film, reference numeral 55 a protection film, and reference numeral 56 a taking electrode. The protection film 55 is to prevent the taking electrode 56 from coming in contact with an electrolyte 4, which is placed on the protection film 55 plus the sensitive film 54.

Such a multi-array sensor can be formed in the following procedures.

An SOS substrate, for example, about 1 (μm) thick and about 20 square (mm) is subjected to thermal oxidation to form a silicon oxide film 50 (nm) thick, and a silicon nitride film about 50 (nm) thick is formed on the silicon oxide film to form an insulator layer 53.

Then a resist film is formed by photolithography at the portions on the insulator layer 53 which are to be formed into sensors, and the other portion is removed by etching to form a laminated structure in the form of an island, which is made up of a silicon layer 52 and the insulator layer 53, on a sapphire substrate (insulating substrate 51). Further, a resist film is formed on the insulator layer 53, excluding its periphery, by photolithography, and the insulator layer 53 is selectively etched to expose the silicon layer 52.

Then an ohmic taking electrode 56 is formed on the silicon layer 52 by evaporation, and the unnecessary part of the aluminium film is removed by lift-off.

After that, a protection film 55 consisting of chemically resistant resist is formed throughout by the spin coating method so that the taking electrode 56 and the electrolyte are not energized. Then single-stranded DNA as a molecule probe is fixed at the portions on the insulator layer 53 which are to be formed into sensitive films 54, and an electrolyte is placed in such a manner as to enclose the sensitive films.

A reference electrode and a counter electrode are provided in the electrolyte and bias voltage and an alternating electric signal are applied to the counter electrode.

If the target DNA hybridizes to the molecule probe, a shift of detected photocurrent is seen in the voltamograph, whereby the DNA can be detected.

In the LAPS method, since the molecule-probe included arrays are read out one by one, it generally gives rise to a problem in that the amount of time spent in measurement increases as the number of arrays increases. However, when using an alternating electric signal as means for stimulating the semiconductor layer, since each array has a taking electrode 56 formed thereon, measurement can be done on multiple arrays simultaneously.

While the molecule detecting sensor for detecting a specific molecule has been described in terms of using a molecule probe having a gene sequence complementary to one that is to be measured to detect a sequence of nucleic acid or the derivative thereof, that is, a gene sequence, this is not intended to limit this invention; and any molecule probes are applicable as long as they can combine with specific molecules, thus the use of the molecule detecting sensor allows the specific molecules as describe above to be detected.

A voltamograph was obtained after the measurements of photocurrent using a molecule detecting sensor for detecting a specific molecule shown in FIG. 25.

The molecule detecting sensor for detecting a specific molecule was formed in the following procedures using an SOS substrate 11a made up of a sapphire substrate 11 and a silicon layer 2 as shown in FIG. 25.

First, an SOS substrate 11a of which silicon layer 2 had a film thickness of 1 (μm) was subjected to thermal oxidation in the oxygen atmosphere to form a silicon oxide layer 3b thickness of 50 (nm), and a silicon nitride layer 3a with a film thickness of 50 (nm) was formed on the silicon oxide layer by the CVD method.

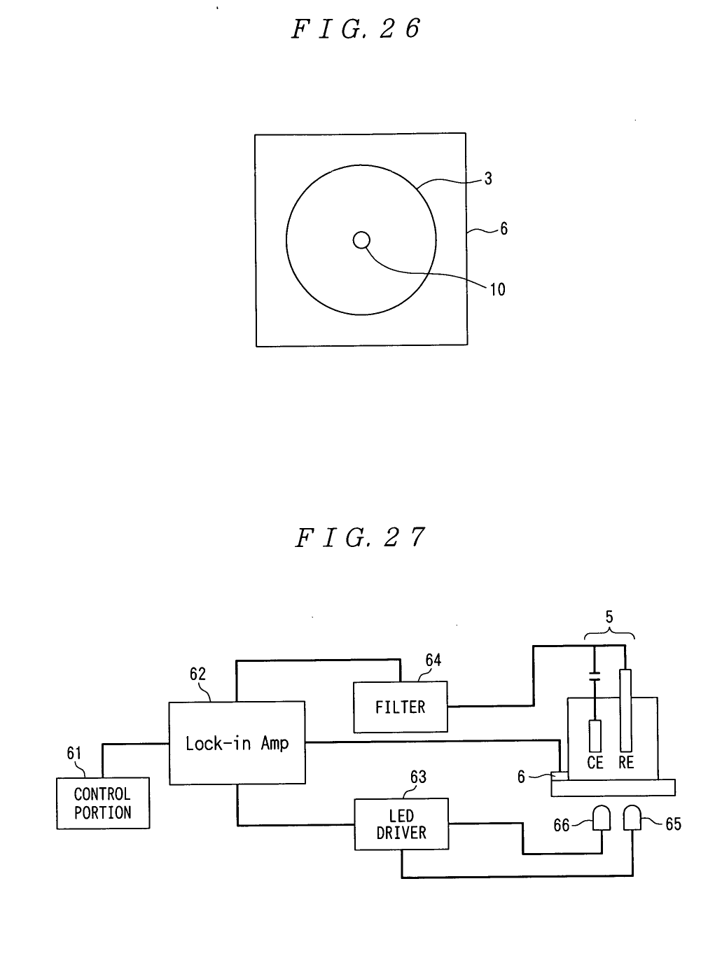

Then an ohmic taking electrode 6was formed on the silicon layer 2. Specifically, a resist film 16 (mmφ) was formed by photolithography nearly in the middle of the silicon nitride layer 3a, and the portion of the silicon nitride layer 3a and silicon oxide layer 3b other than that protected with the resist film was removed by RIE to expose the portion of the silicon layer 2 on which the taking electrode was to be formed. Then aluminium was deposited on the exposed portion of the silicon layer 2, and the resist film as well as the unnecessary part of the aluminium film was removed by lift-off. Thus, as shown in FIG. 26, the molecule detecting sensor was obtained in which the insulator layer 3 made up of the silicon nitride layer and the silicon oxide layer was arranged in the middle of the silicon layer 2 and the aluminium electrode as the taking electrode 6 was formed in such a manner as to enclose the insulator layer 3.

Then a single-stranded DNA as a molecule probe was fixed on the insulator layer 3 to form a sensitive film 10. The sensitive film 10 was formed in the middle of the insulator layer 3 to be about 2 (mmφ) in size. Thus, the sensitive film 10 consisting of a DNA probe was formed in the middle of the insulator layer 3, as shown in the top view of FIG. 26.

A container 4a for containing an electrolyte 4 was then contact bonded to the molecule detecting sensor so that the insulator layer 3 and the electrolyte 4 could come in contact with each other. Here the contact bonding was performed using an O ring (not shown in the drawing) so that the electrolyte 4 did not come in contact with the taking electrode 6.

The container 4a was filled with the electrolyte 4, a reference electrode 5 was put into the electrolyte 4, and bias voltage BV was applied between the taking electrode 6 and the reference electrode 5. Then the back of the SOS substrate 3 was exposed to light via a shielding plate not shown in the drawing using blue LEDs 65, 66 of wavelength 475 (nm), and photocurrent induced was measured through signal processing at a photocurrent signal processing portion 7 provided between the taking electrode 6 and the reference electrode 5. The pore diameter of the shielding plate was 0.8 (mmφ). The measurements were made on the sensitive film 10 portion, where the single-stranded DNA was fixed, and the portion other than the sensitive film 10 portion, while changing the bias voltage BV from negative to positive. The photocurrent obtained was recorded to obtain a voltamograph.

Then the target DNA was put into the electrolyte 4 and allowed to undergo hybridization, and measurements were made after performing light illumination in the same manner as above.

The measurements were made twice before and after the hybridization, respectively.

FIG. 27 is a schematic block diagram showing the photocurrent signal processing portion 7. A modulated light is generated by controlling a lock-in amplifier 62 with a control portion 61 consisting of a personal computer etc. A LED driver 63 drives the LED 65, which is used to illuminate the sensitive film 10 portion, and the LED 66, which is used to illuminate the portion other than the sensitive film 10 portion, according to the modulated light. After a photocurrent signal is input into the control portion 61 through a filter 64 and the lock-in amplifier 62, the quantity of photocurrent induced can be measured. A voltamograph is made up of the photocurrent values measured while varying bias voltage BV and the values of the varied bias voltage BV.

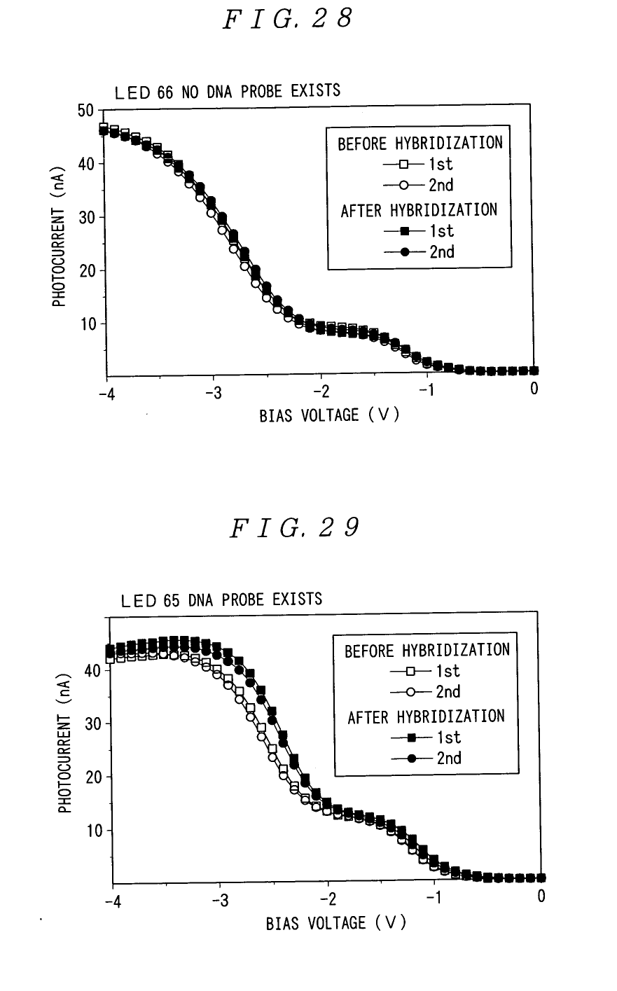

FIGS. 28 and 29 show examples of voltamographs obtained as above, respectively. In each voltamograph, bias voltage (V) is plotted in abscissa and the detected values of photocurrent (nA) in ordinate.

FIG. 28 shows the measured results obtained when driving the LED 66, which is used to illuminate the portion other than the sensitive film 10 portion. It is apparent from the figure that there existed almost no changes before and after hybridization.

FIG. 29 shows the measured results obtained when driving the LED 65, which is used to illuminate the sensitive film 10 portion. It is apparent from the figure that the voltamograph shifts to the plus side by about 200 (mV) at the sensitive film 10 portion due to the DNA hybridization.

Thus, it has been as certained that if there exists a target DNA at the portion where a single-stranded DNA as a molecule probe is fixed and both hybridize to each other, changes are caused in photocurrent induced, and the target DNA can be detected by reading the changes or the difference in photocurrent from the reference portion where no DNA probe is fixed.

In this case, if bias voltage is fixed at −3 to −2.5 (V), the hybridized DNA can be detected by detecting the increase in photocurrent value due to the hybridization.

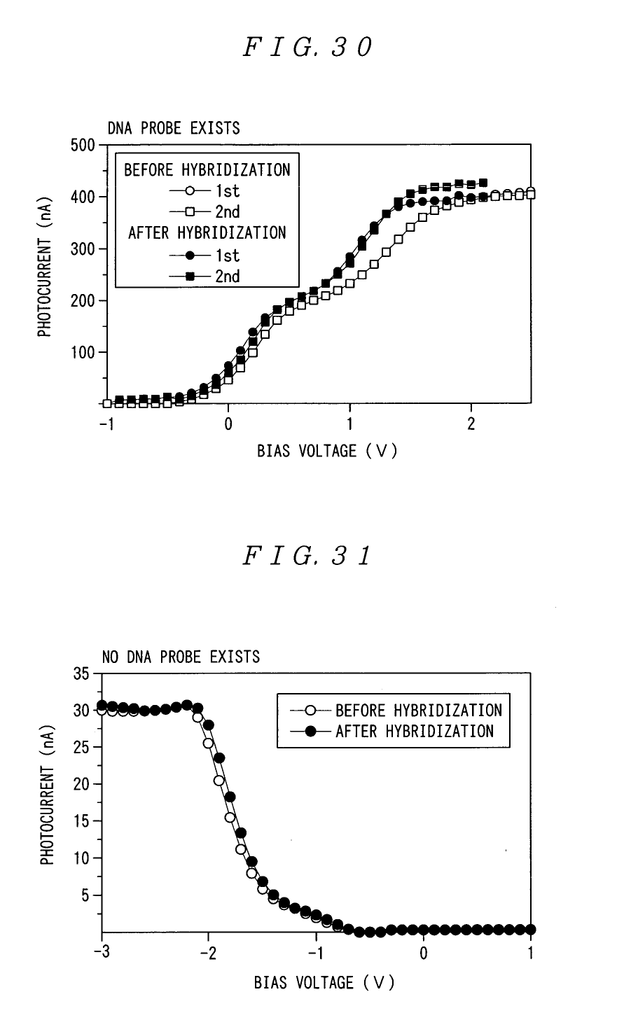

FIG. 30 is one example of the voltamographs obtained when a DNA is hybridized in the above described molecule detecting sensor, which uses an SOS substrate including a silicon layer of 2 (μm) thick, by driving the LED 65 which is used to illuminate the sensitive film 10 portion. It is apparent from the figure that the voltamograph shifts to the minus side by about 200 (mV) at the sensitive film 10 portion due to the DNA hybridization. Accordingly, in this case, if bias voltage is fixed at about 1 to about 1.5 (V), the hybridized DNA can be detected by detecting the decrease in photocurrent value due to the hybridization.

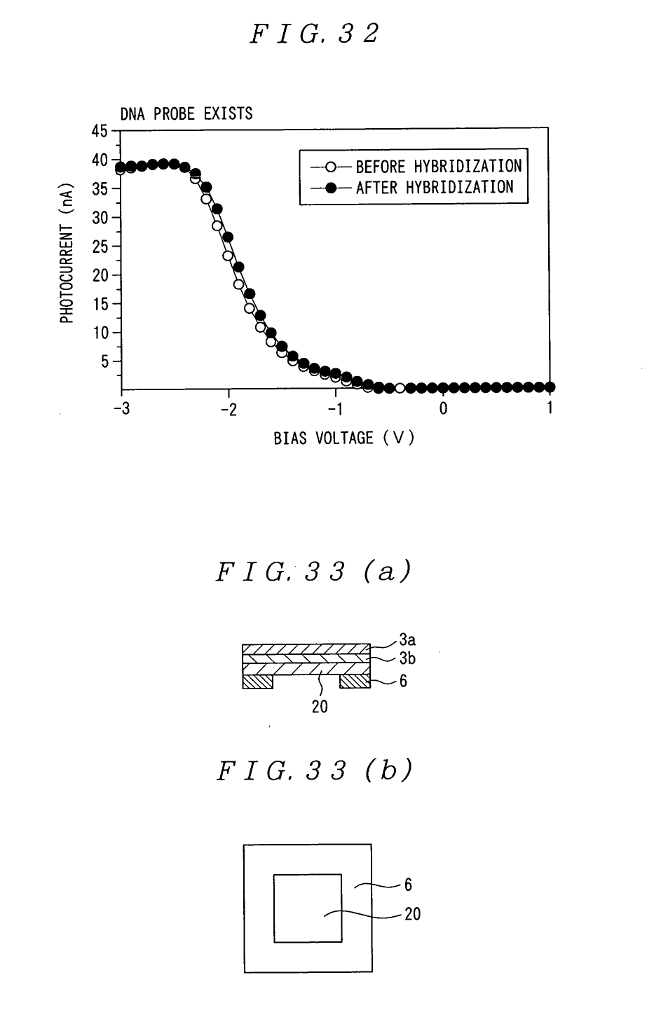

FIGS. 31 and 32 are examples of the voltamographs obtained when a DNA is hybridized in the above described molecule detecting sensor, which uses a bulk silicon substrate instead of an SOS substrate, in the same manner as above.

The above molecule detecting sensor using a bulk silicon layer was formed in the following procedure.

As shown in the side view of FIG. 33(a) and the bottom view of FIG. 33(b), a silicon substrate 20 which is 2.54 (cmφ) in size and 280 (nm) in thickness was cut down 20 square (mm), and subjected to thermal oxidation in the oxygen atmosphere to form a silicon oxide film 3b with a film thickness of 50 (nm). Further, a silicon nitride film 3a with a film thickness of 50 (nm) was formed on the silicon oxide film 3b by the CVD.

Then, an ohmic taking electrode 6 was formed on the other side of the silicon substrate. Specifically, a resist film 16 (mmφ) was formed in the middle of the silicon substrate by photolithography, aluminium was deposited thereon, and the resist film as well as the unnecessary part of the aluminium film was removed.

Thus, as shown in FIG. 33, a molecule detecting sensor 10 was obtained in which a taking electrode 6 consisting of aluminium was formed on its back in such a manner as to enclose a light entering portion 16 (mmφ).

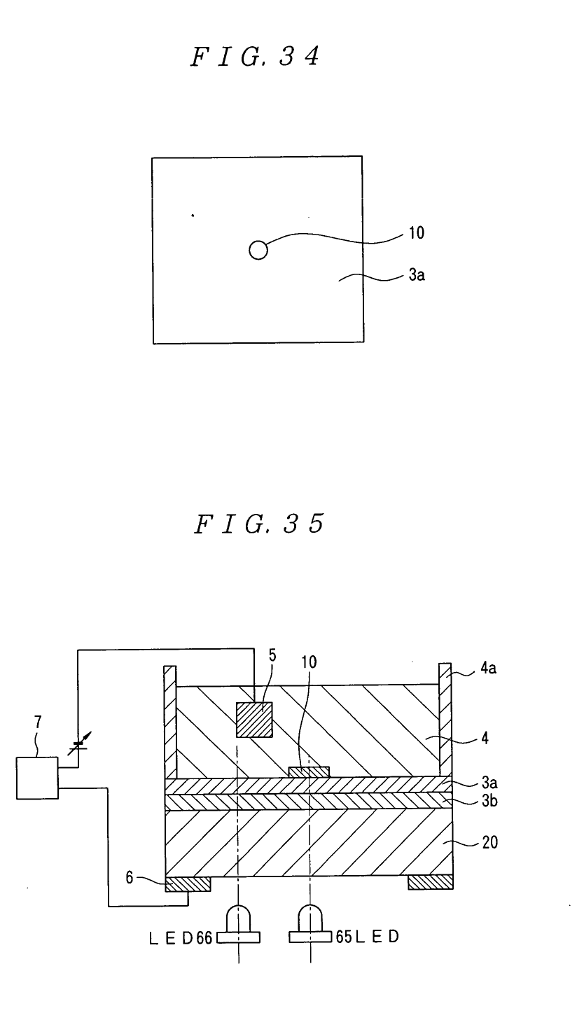

And, as shown in FIG. 34, a single-stranded DNA as a molecule probe was fixed on an insulator layer, that is, on the silicon nitride film 3a to form a sensitive film 10 consisting of the molecule probe, as shown in 34. The sensitive film 10 was fixed in the middle of the insulator layer to be about 2 (mmφ) in size.

With this molecule detecting sensor, photocurrent was detected, in the same manner as the above described examples, as shown in FIG. 35, by putting a reference electrode 5 into an electrolyte 4 in a container 4a, applying bias voltage BV between the taking electrode 6 and the reference electrode 5, performing light illumination using red LEDs 65, 66 of wave length 945 (nm), and processing the obtained photocurrent signal at a photocurrent signal processing portion 7 which is provided between the taking electrode 6 and the reference electrode 5. In this case, too, photocurrent measurements were made on the portion where a single-stranded DNA was fixed, that is, on the probe portion and on the portion other than the probe portion while changing bias voltage BV from negative to positive. Thus, the voltamographs of FIGS. 31 and 32 were obtained.

It is apparent from FIGS. 31 and 32 that there existed almost no changes before and after the hybridization. In other words, with the above described LAPS which used a bulk silicon substrate, a DNA, which had not been modified, could not be detected.

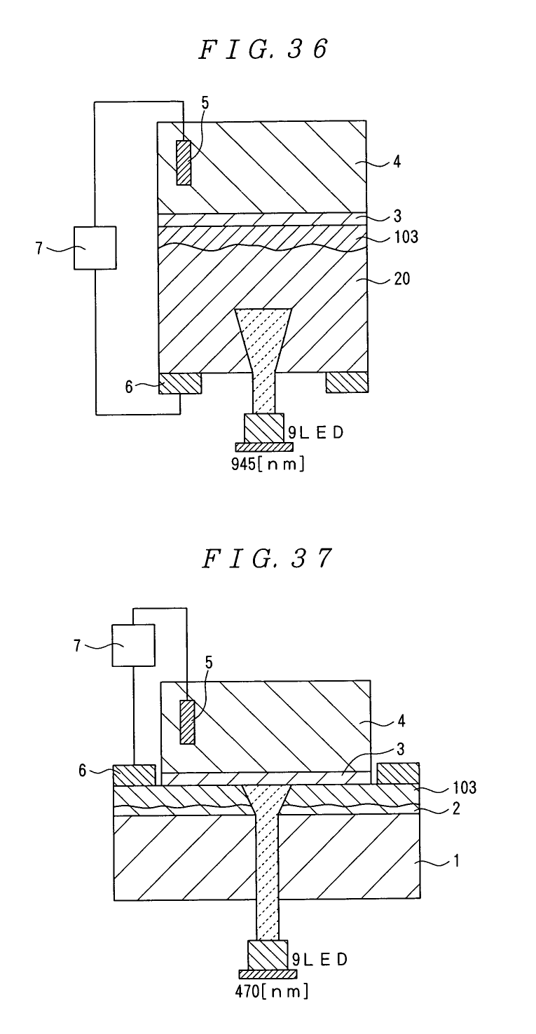

FIGS. 36 and 37 illustrate the states in which photocurrent is induced in a molecule detecting sensor using a bulk silicon substrate and in which photocurrent is induced in a molecule detecting sensor using an SOS substrate, respectively.

As shown in FIG. 36, in the molecule detecting sensor which is formed using a bulk silicon substrate and a silicon substrate 20, an insulator layer 3, and an electrolyte 4 stacked in this order, even if light illumination is performed from the silicon substrate 20 side using infrared light of which wavelength is about 945 (nm), the infrared light penetrates only about 60 (μm) deep. To induce photocurrent, carriers need to be diffused up to the depletion layer 103, however some carriers are lost before reaching the layer due to their recombination etc.

On the other hand, as shown in FIG. 37, in the molecule detecting sensor which is formed using an SOS substrate and a light transmitting substrate 1, a silicon layer 2, an insulator layer 3, and an electrolyte 4 stacked in this order, the depletion layer 103 spreads as far as the vicinity of the light transmitting substrate 1-silicon layer 2 interface, according to conditions, and optically pumped carriers can be generated in the depletion layer 103. Accordingly, almost all the optically pumped carriers can be taken as a drift current by the force of the electric field.

Thus, even when using a bulk silicon substrate in the molecule detecting sensor, if optically pumped carriers can be generated in the depletion layer 103, a DNA, to which labeling has not been adopted, can be detected.

In order to generate optically pumped carriers in the depletion layer and its vicinities using a bulk silicon substrate, light illumination should be performed from the insulator layer side to expose the insulator surface to light.

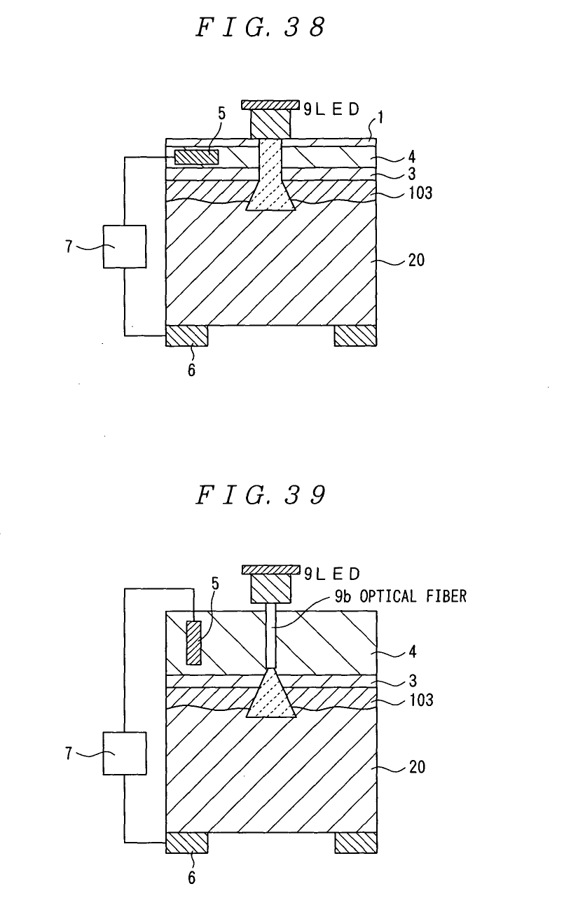

FIG. 38 is a schematic block diagram showing one example of the molecule detecting sensors using a bulk silicon substrate.

This type of molecule detecting sensor is formed in the following procedure.

First, a silicon substrate 20 which is 2.54 (cmφ) in size is cut down 20 square (mm), and subjected to thermal oxidation in the oxygen atmosphere to form a silicon oxide film with a film thickness of 50 (nm). Further, a silicon nitride film with a film thickness of 50 (nm) is formed on the silicon oxide film by the CVD to form an insulator layer 3.

Then a single-stranded DNA as a molecule probe is fixed on the insulator layer 3, and an electrolyte 4 is placed on the insulator layer 3 including the molecule probe. The thinner the electrolyte 4 is, the more preferable it is, and preferably its thickness is about 1 (μm) to 10 (mm).

A light transmitting substrate 1 is placed on the electrolyte 4 in such a manner as to put the electrolyte 4 between the insulator layer 3 and itself.

A reference electrode 5 and a counter electrode, not shown in the drawing, are then provided in the electrolyte 4, and an ohmic electrode is formed as a taking electrode 6.

Then, voltage is applied between the taking electrode 6 and the reference electrode 5 in the same manner as above, light illumination is performed from the light transmitting substrate 1, using the light emitting diode 9, and the photocurrent induced by the light illumination is measured through a photocurrent signal processing portion 7.

The wavelength of the light source is set depending on the concentration of the impurity doped in the silicon substrate 20, and such a wavelength as permits light to penetrate deeper than the maximum spread of the depletion layer 103 created in the silicon substrate 20 is used. From the viewpoint of sensitivity, the wavelength is more preferable which permits the penetration depth of the light and the maximum spread of the depletion layer 103 to correspond to each other.

Accordingly, in this case, too, optically pumped carriers can be generated in the depletion layer 103, and changes in photocurrent value depending on the chemical quantity and the substance in the electrolyte 4 can be detected; thus, a DNA, which has not been modified, can be detected like when using a molecule detecting sensor which uses an SOS substrate.

This invention is not limited to the above described examples; for example, as shown in FIG. 39, light may be introduced into an electrolyte 4 from its outside through a optical fiber etc. having been immersed in the electrolyte 4. Furthermore, a waterproofed light source maybe permeated in the electrolyte to introduce light near a boundary surface between the semiconductor layer and the insulator layer 3. In this case, it does not matter how thick the electrolyte is.

In the above embodiment, desirably the wavelength of the light used and the concentration of the impurity doped in the semiconductor layer are set so that the penetration depth of the light becomes almost equal to the maximum spread of the depletion layer. In other words, desirably they are set so that they satisfy the following equation (2), where the left side represents the penetration depth of light and the right side the maximum spread of depletion layer.

λ/2πk≈(2εₛ·ε₀·(2Φ_{f}))/qN)^{1/2} (2)

Φᵣ=(k_{B}·T/q)·1n(N/ni)

wherein λ represents the wavelength of incident light, k the extinction coefficient, εₛ the relative dielectric constant of silicon, ε₀ the relative dielectric constant of vacuum, q charge, N the concentration of impurity, Φ_{f} Fermi potential, k_{B} Boltzman constant, T the absolute temperature, and ni the intrinsic carrier concentration.

Since the most important thing is to allow optically pumped carriers to be generated in the depletion layer, as described above, the depth of light penetration and the maximum spread of the depletion layer should be made almost equal to each other, and desirably the depth of light penetration and the maximum spread of the depletion layer are preferably set so that they are almost equal to each other and the depth of light penetration is larger than the maximum spread of the depletion layer.

Accordingly, if the wavelength of incident light and the concentration of the impurity are set, taking into account the optical absorption and the like of electrolyte, so that they satisfy the above equation (2), optically pumped carriers can be generated in the depletion layer, as described above; thus, a DNA, which has not been modified, can be detected like when using a molecule detecting sensor which uses an SOS substrate.

In the above embodiment, while the molecule detecting sensor which uses the silicon layer on a light transmitting substrate has been described in terms of the case where stimulation, such as light illumination, is provided from the light transmitting substrate side, this is not intended to limit this invention, and the stimulation may be provided from the electrolyte side.

Further, in the above embodiment, while the molecule detecting sensor which uses the silicon layer on a light transmitting substrate has been described in terms of the case where the insulator substrate such as a sapphire substrate is used as a light transmitting substrate, this is not intended to limit this invention; and, a light transmitting conductive substrate can also be used as a light transmitting substrate when adopting light illumination as means for providing stimulation, this is not applicable when adopting an alternating electrical signal as means for providing stimulation, though.

Further, in the above molecule detecting sensor for detecting specific molecule, the silicon layer on a carrier substrate which uses the above first to fourth embodiments can also be used. With the silicon layer on a carrier substrate formed described above, detecting a specific molecule can be performed with higher accuracy.

Industrial Application

As described so far, according to this invention, since the photocurrent characteristics of the SOI substrate made up of a semiconductor layer and a light transmitting layer can be improved, a molecule detecting sensor of excellent performance, for example, of high resolution, high sensitivity and high stability can be obtained. Further, if such a molecule detecting sensor is used to construct an array sensor, an array sensor of excellent performance, for example, of high resolution, high sensitivity and high stability can be obtained.

Further, according to this invention, the bonding between a molecule probe and a specific molecule can be detected by detecting a change in electric characteristics induced by stimulating a semiconductor substrate or a semiconductor layer; therefore, detection of a specific molecule can be performed more easily or rapidly without adopting complicated procedures such as labeling.

1. A molecule detecting sensor which is designed to comprise a semiconductor layer and an insulator layer stacked in this order on a light transmitting substrate and an electrolyte placed on the insulator layer and to detect molecules in the steps of:
illuminating the semiconductor layer with light from the light transmitting substrate side; and
quantitatively determining the electrolyte based on the photocurrent induced in the semiconductor layer through the illumination of light,
characterized in that said semiconductor layer is a single crystal silicon layer and the FWHM of the X-ray diffraction rocking curve of the (004) plane of the single crystal silicon layer is 1000 (arcsec) or less or the crystal defect density of the same is 1×10⁸ (cm⁻²) or less.

2. The molecule detecting sensor according to claim 1,
characterized in that said single crystal silicon layer has a surface roughness of 4 (nm) or less.

3. The molecule detecting sensor according to claim 1 or 2,
characterized in that said single crystal silicon layer has a film thickness of 1 (nm) or more and 1×10⁵ (nm) or less:

4. A molecule detecting sensor which is designed to comprise a semiconductor layer and an insulator layer stacked in this order on a light transmitting substrate and an electrolyte placed on the insulator layer and to detect molecules in the steps of:
illuminating the semiconductor layer with light from the light transmitting substrate side; and
quantitatively determining the electrolyte based on the photocurrent induced in the semiconductor layer through the illumination of light,
characterized in that there exists heavily doped impurity in the semiconductor layer in the vicinity of the semiconductor layer-light transmitting substrate interface.

5. The molecule detecting sensor according to claim 4,
characterized in that as the heavily doped impurity, 1×10¹⁷ to 1×10²⁰ (cm⁻³) of impurity is added.

6. A molecule detecting sensor which is designed to comprise a semiconductor layer and an insulator layer stacked in this order on a light transmitting substrate and an electrolyte placed on the insulator layer and to detect molecules in the steps of:
illuminating the semiconductor layer with light from the light transmitting substrate side; and
quantitatively determining the electrolyte based on the photocurrent induced in the semiconductor layer through the illumination of light,
characterized in that a transparent conductive film is provided between the semiconductor layer and the light transmitting substrate.

7. A molecule detecting sensor which is designed to comprise a semiconductor layer and an insulator layer stacked in this order on a light transmitting substrate and an electrolyte placed on the insulator layer and to detect molecules in the steps of:
illuminating the semiconductor layer with light from the light transmitting substrate side; and
quantitatively determining the electrolyte based on the photocurrent induced in the semiconductor layer through the illumination of light,
characterized in that said light transmitting substrate is an electro-conductive substrate.

8. The molecule detecting sensor according to any one of claims 1 to 7, characterized in that an antireflection film is provided at least either between the semiconductor layer and the light transmitting substrate or on the light transmitting substrate surface exposed to light.

9. The molecule detecting sensor according to claim 8, characterized in that said thickness of said antireflection film is set according to the wavelength of the illumination light.

10. The molecule detecting sensor according to any one of claims 1 to 9, characterized in that the thickness of said semiconductor layer is set according to the wavelength of the illumination light.

11. The molecule detecting sensor according to any one of claims 1 to 10, characterized in that said light transmitting substrate is a single crystal oxide substrate or a glass substrate containing SiO₂.

12. The molecule detecting sensor according to claim 11, characterized in that said single crystal oxide substrate is a sapphire substrate.

13. The molecule detecting sensor according to any one of claim 1, claim 2 and claims 4 to 12, characterized in that the thickness of said semiconductor layer is 10 (μm) or less.

14. A method of manufacturing a molecule detecting sensor which is designed to include a semiconductor layer and an insulator layer stacked in this order on a light transmitting substrate and an electrolyte placed on the insulator layer and to detect molecules in the steps of:
illuminating the semiconductor layer with light from the light transmitting substrate side; and
quantitatively determining the electrolyte based on the photocurrent induced in the semiconductor layer through the illumination of light,
characterized by comprising:
a film forming step of forming a first silicon layer on the light transmitting substrate;
an oxidizing step of oxidizing exclusively the top portion of the first silicon layer by heat treating the same in the oxidizing atmosphere; and
a removing step of removing the silicon oxide film formed in the oxidizing step and it uses the first silicon layer formed after the removing step as the semiconductor layer.

15. The method of manufacturing a molecule detecting sensor according to claim 14, characterized in that said method further comprises an epitaxy step of growing a second silicon layer by epitaxy on the first silicon layer formed after the removing step and uses the laminated structure made up of the first and second silicon layers as the semiconductor layer.

16. The method of manufacturing a molecule detecting sensor according to claim 14 or 15, characterized by further comprising are crystallizing step, right after the film forming step, of recrystallizing the first silicon layer in the sub-steps of: implanting silicon ions into the first silicon layer to turn the same amorphous in the vicinity of the first silicon layer-light transmitting substrate interface; and heat treating the amorphous silicon layer.

17. The method of manufacturing a molecule detecting sensor according to claim 15, characterized in that it repeats the oxidizing, removing and epitaxy steps two times or more for the second silicon layer having been formed through the epitaxy step, considering the same as the first silicon layer in the oxidizing step, and uses the laminated structure made up of the silicon layers thereby formed as the semiconductor layer.

18. A method of manufacturing a molecule detecting sensor which is designed to comprise a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light,
characterized in that said method comprises:
a film forming step of forming a first silicon layer on the light transmitting substrate;
a first recrystallizing step of recrystallizing the first silicon layer in the sub-steps of implanting silicon ions into the first silicon layer to turn the same amorphous in the vicinity of the first silicon layer-light transmitting substrate interface and heat treating the amorphous silicon layer;
an oxidizing step of oxidizing exclusively the top portion of the first silicon layer by heat treating the first silicon layer in the oxidizing atmosphere after the first recrystallizing step;
a removing step of removing the silicon oxide film formed in the oxidizing step;
an epitaxy step of growing a second silicon layer by epitaxy on the first silicon layer left after the removing step; and
a second recrystallizing step of recrystallizing the laminated structure made up of the first and second silicon layers in the sub-steps of implanting silicon ions into the laminated structure to turn the same amorphous in the vicinity of the laminated structure-light transmitting substrate interface and heat treating the amorphous laminated structure and
it uses the laminated structure of the silicon layers after the second recrystallizing step as the semiconductor layer.

19. The method of manufacturing a molecule detecting sensor according to claim 18, characterized in that said method comprises an epitaxy step of growing a third silicon layer by epitaxy on the second silicon layer after the second recrystallizing step and uses the laminated structure made up of the silicon layers including the third silicon layer as the semiconductor layer.

20. The method of manufacturing a molecule detecting sensor according to claim 18, characterized in that said method comprises, after the second recrystallizing step, an oxidizing step of oxidizing exclusively the top portion of the second silicon layer by heat treating the same in the oxidizing atmosphere; a removing step of removing the silicon oxide film formed by the oxidation; and an epitaxy step of growing a third silicon layer by epitaxy on the second silicon layer left after the removing step and uses the laminated structure made up of the silicon layers including the third silicon layer as the semiconductor layer.

21. A method of manufacturing a molecule detecting sensor which is designed to comprise a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light,
characterized in that said method comprises the steps of:
implanting hydrogen or rare gas ions into the surface of a single crystal silicon substrate to form an ion diffusion region of hydrogen or rare gas therein;
bonding the ion diffusion region of the single crystal silicon substrate and the light transmitting substrate together;
causing the single crystal silicon substrate to cleave in the ion diffusion region by heat treatment after the bonding step, to form a single crystal silicon layer on the light transmitting substrate; and
planarizing the cleaved surface of the single crystal silicon layer by polishing and that it uses the planarized single crystal silicon layer as the semiconductor layer.

22. A method of manufacturing a molecule detecting sensor which is designed to comprise a semiconductor layer and an insulator layer stacked in this order on a light transmitting substrate and an electrolyte placed on the insulator layer and to detect molecules in the steps of: illuminating the semiconductor layer with light from the light transmitting substrate side; and quantitatively determining the electrolyte based on the photocurrent induced in the semiconductor layer through the illumination of light,
characterized in that said method comprises the steps of: anodizing the surface of a single crystal silicon substrate to form a porous single crystal silicon layer; annealing the porous single crystal silicon layer in the hydrogen atmosphere to make the uppermost surface of the same a single crystal silicon layer on which another single crystal silicon layer is grown by epitaxy; bonding the light transmitting substrate to the surface of the grown single crystal layer; removing the single crystal silicon substrate and the porous single crystal silicon layer; and planarizing the surface of the single crystal silicon layer having been exposed after the removing step and that it uses the planarized single crystal silicon layer as the semiconductor layer.

23. The method of manufacturing a molecule detecting sensor according to any one of claims 14 to 22,
characterized by further comprising a step of arranging a transparent conductive film between the semiconductor layer and the light transmitting substrate.

24. The method of manufacturing a molecule detecting sensor according to any one of claims 14 to 23,
characterized by further comprising a step of arranging a heavily doped impurity in the semiconductor layer in the vicinity of the semiconductor layer-light transmitting substrate interface.

25. The method of manufacturing a molecule detecting sensor according to any one of claims 14 to 24,
characterized by further comprising a step of forming an antireflection film between the semiconductor layer and the light transmitting substrate.

26. The method of manufacturing a molecule detecting sensor according to any one of claims 14 to 25,
characterized by further comprising a step of forming an antireflection film on the light transmitting substrate surface exposed to light.

27. The method of manufacturing a molecule detecting sensor according to claim 25 or 26,
characterized in that the thickness of the antireflection film is set according to the wavelength of the illumination light.

28. The method of manufacturing a molecule detecting sensor according to any one of claims 14 to 27,
characterized in that the light transmitting substrate is a single crystal oxide substrate or a glass substrate containing SiO₂.

29. The method of manufacturing a molecule detecting sensor according to claim 28,
characterized in that the single crystal oxide substrate is a sapphire substrate.

30. A molecule detecting sensor which is designed to comprise a semiconductor layer and an insulator layer stacked in this order, a plurality of sensitive films formed on the insulator layer and an electrolyte placed on the sensitive films and to detect molecules in the steps of: illuminating the semiconductor layer with light from its back; detecting the photocurrent induced in the semiconductor layer through the illumination of light using electrodes provided in the electrolyte as well as on the semiconductor layer; and quantitatively determining the electrolyte based on the detected photocurrent,
characterized in that the plurality of sensitive films are positioned so that they are the same distance from the electrode provided on the semiconductor layer.

31. The molecule detecting sensor according to claim 30,
characterized in that the electrode on the semiconductor layer is provided around the periphery of the region corresponding to the lower part of the electrolyte of the semiconductor layer and the sensitive films are provided in a plurality of linear arrays at the positions which are the same distance from the electrode provided on the semiconductor layer.

32. A molecule detecting sensor comprising: an insulator layer superposed on a semiconductor layer; a molecule probe which is fixed on the insulator layer and combines with a specific molecule; an electrolyte arranged on the insulator layer containing at least the molecule probe; and electrical characteristic detecting means for detecting electrical characteristics induced by stimulating the semiconductor,
characterized in that it detects the specific molecule n the electrolyte by detecting the changes in the electrical characteristics due to the bonding of the molecule probe to the specific molecule in the electrolyte.

33. The molecule detecting sensor according to claim 32,
characterized in that said semiconductor layer is provided on a carrier substrate.

34. The molecule detecting sensor according to claim 32 or 33, characterized in that said specific molecule is a nucleic acid or the derivative thereof.

35. The molecule detecting sensor according to any one of claims 32 to 34,
characterized in that a plurality of molecule probes are arranged on the insulator layer.

36. The molecule detecting sensor according to any one of claims 32 to 35,
characterized in that molecule detection is performed based on the results of the differential measurements made for the sites where the molecule probe exists and where no molecule probe exists.

37. The molecule detecting sensor according to any one of claims 32 to 36,
characterized in that the electrical characteristic detecting means is constructed so that it applies an electric field between the electrolyte and the semiconductor layer to bond the molecule probe and the specific molecule together and applies an electric field again to release the molecule probe and the specific molecule from their incomplete bonding due to the first application of electric field.

38. The molecule detecting sensor according to any one of claims 32 to 37,
characterized in that a bonding molecule which can combine with the specific molecule, but is different from the molecule probe is introduced into the electrolyte.

39. The molecule detecting sensor according to claim 38,
characterized in that said bonding molecule is an intercalator.

40. The molecule detecting sensor according to claim 38,
characterized in that said bonding molecule is a protein which can combine with a nucleic acid or the derivative thereof.

41. The molecule detecting sensor according to claim 40,
characterized in that said protein is an antibody which can combine with a nucleic acid or the derivative thereof.

42. The molecule detecting sensor according to claim 38,
characterized in that said bonding molecule is a nucleic acid or the derivative thereof.

43. The molecule detecting sensor according to any one of claims 38 to 42,
characterized in that said bonding molecule is modified with urease.

44. The molecule detecting sensor according to any one of claims 38 to 42,
characterized in that said bonding molecule is modified with ferrocene.

45. The molecule detecting sensor according to any one of claims 33 to 44,
characterized in that said carrier substrate is a light transmitting substrate.

46. The molecule detecting sensor according to any one of claims 32 to 45,
characterized in that said semiconductor layer is a single crystal silicon layer.

47. The molecule detecting sensor according to claims 46, characterized in that in the single crystal silicon layer, the FWHM of the X-ray diffraction rocking curve of its (004) plane is 1000 (arcsec) or less and its crystal defect density is 1×10⁸ (cm⁻²) or less.

48. The molecule detecting sensor according to claim 46 or 47,
characterized in that the surface roughness of said single crystal silicon layer is 4 (nm) or less.

49. The molecule detecting sensor according to any one of claims 32 to 48,
characterized in that the thickness of said semiconductor layer is set according to the wavelength of the illumination light.

50. The molecule detecting sensor according to any one of claims 32 to 49,
characterized in that the thickness of said semiconductor layer is 10 (μm) or less.

51. The molecule detecting sensor according to any one of claims 45 to 50,
characterized in that the reexists heavily doped impurity with a concentration of 1×10¹⁷ to 1×10²⁰ (cm⁻³) in the semiconductor layer in the vicinity of the semiconductor layer-light transmitting substrate interface.

52. The molecule detecting sensor according to any one of claims 45 to 50,
characterized in that a transparent conductive film is provided between said semiconductor layer and said light transmitting substrate.

53. The molecule detecting sensor according to any one of claims 45 to 52,
characterized in that an antireflection film is provided at least either between said semiconductor layer and said light transmitting substrate or on the light transmitting substrate surface exposed to light.

54. The molecule detecting sensor according to claims 53,
characterized in that the thickness of said antireflection film is set according to the wavelength of the illumination light.

55. The molecule detecting sensor according to any one of claims 33 to 54,
characterized in that said carrier substrate and said semiconductor layer are an SOS substrate consisting of a sapphire single crystal substrate and a single crystal silicon layer.

56. The molecule detecting sensor according to any one of claims 33 to 55,
characterized in that said carrier substrate is an electro-conductive substrate.

57. The molecule detecting sensor according to any one of claims 33 to 56,
characterized in that said stimulation is a modulated electromagnetic wave.

58. The molecule detecting sensor according to any one of claims 33 to 56,
characterized in that the stimulation is a modulated electric signal and the carrier substrate an insulating substrate.

59. A method of detecting molecules,
characterized in that it detects a specific molecule in an electrolyte under the condition that a semiconductor layer and an insulator layer are stacked in this order on a carrier substrate, a molecule probe, which combines with the specific molecule, is fixed on the insulator layer and the electrolyte arranged on the insulator layer which includes at least the molecule probe, by: detecting electric characteristics induced through the stimulation of the semiconductor layer; and detecting the changes in the electric characteristics due to the bonding of the molecule probe to the specific molecule in the electrolyte.

60. A method of detecting molecules,
characterized in that it detects a specific molecule in an electrolyte under the condition that a semiconductor layer and an insulator layer are stacked in this order on a carrier substrate, a molecule probe, which can combine with the specific molecule, is fixed on the insulator layer and the electrolyte is arranged on the insulator layer which includes at least the molecule probe, by: introducing into the electrolyte a bonding molecule which can combine with the specific molecule but is different from the molecule probe; detecting electronic characteristics induced through the stimulation of the semiconductor layer; and detecting the changes in the electric characteristics due to the bonding of the molecule probe to the specific molecule in the electrolyte as well as the bonding of the specific molecule with the bonding molecule.

61. A molecule detecting sensor which is designed to include: an insulator layer superposed on a semiconductor layer; a molecule probe which is fixed on the insulator layer and combines with a specific molecule; an electrolyte placed on the insulator layer which includes at least the molecule probe; and electric characteristic detecting means for detecting the electric characteristics induced through the stimulation of the semiconductor layer and to detect the specific molecule in the electrolyte by detecting the changes in the electric characteristics due to the bonding of the molecule probe to the specific molecule in the electrolyte,
characterized in that the stimulation of the semiconductor layer is provided from the electrolyte side.

62. The molecule detecting sensor according to claim 61,
characterized in that the specific molecule is a nucleic acid or the derivative thereof.

63. The molecule detecting sensor according to claim 61 or 62,
characterized in that a plurality of molecule probes are arranged on the insulator layer.

64. The molecule detecting sensor according to any one of claims 61 to 63,
characterized in that molecule detection is performed based on the results of the-differential measurements made for the sites where the molecule probe exists and where no molecule probe exists.

65. The molecule detecting sensor according to any one of claims 61 to 64,
characterized in that the electrical characteristic detecting means is constructed so that it applies an electric field between the electrolyte and the semiconductor layer to bond the molecule probe and the specific molecule together and applies an electric field again to release the molecule probe and the specific molecule from their incomplete bonding due to the first application of electric field.

66. The molecule detecting sensor according to any one of claims 61 to 65,
characterized in that a bonding molecule which can combine with the specific molecule but is different from the molecule probe is introduced into the electrolyte.

67. The molecule detecting sensor according to claim 66,
characterized in that the bonding molecule is an intercalator.

68. The molecule detecting sensor according to claim 66,
characterized in that the bonding molecule is a protein which can combine with a nucleic acid or the derivative thereof.

69. The molecule detecting sensor according to claim 68,
characterized in that the protein is an antibody which can combine with a nucleic acid or the derivative thereof.

70. The molecule detecting sensor according to claim 66,
characterized in that the bonding molecule is a nucleic acid or the derivative thereof.

71. The molecule detecting sensor according to any one of claims 66 to 70,
characterized in that the bonding molecule is modified with urease.

72. The molecule detecting sensor according to any one of claims 66 to 70,
characterized in that the bonding molecule is modified with ferrocene.
